(19) United States
(12) Patent Application Publication
(10) Pub. No.: US 2005/0187288 A1
(43) Pub. Date: Aug. 25, 2005

(54) Beta-lapachone and methods of treating cancer

(76) Inventor: Chiang Li, West Roxbury, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/846,980

(22) Filed: May 14, 2004

Related U.S. Application Data
(60) Provisional application No. 60/545,915, filed on Feb. 20, 2004.
(60) Provisional application No. 60/545,916, filed on Feb. 20, 2004.
(60) Provisional application No. 60/545,950, filed on Feb. 20, 2004.
(60) Provisional application No. 60/545,917, filed on Feb. 20, 2004.
(60) Provisional application No. 60/545,914, filed on Feb. 20, 2004.

(51) Int. Cl. A61K 31/353

(52) U.S. Cl. 514/455

(57) ABSTRACT

The present invention provides for methods that utilize agents effective in the treatment of cancerous and pre-cancerous conditions. Moreover, the present invention provides agents capable of acting as an inhibitor of cell proliferation.

### RELATED APPLICATIONS

This application claims priority to U.S. Ser. No. 60/545,915, filed on Feb. 20, 2004; U.S. Ser. No. 60/545,916, filed on Feb. 20, 2004; U.S. Ser. No. 60/545,950, filed on Feb. 20, 2004; U.S. Ser. No. 60/545,917, filed on Feb. 20, 2004; U.S. Ser. No. 60/545,914, filed on Feb. 20, 2004, each of which is incorporated herein by reference in their entireties.

### BACKGROUND OF THE INVENTION

Cancer cells are by definition heterogeneous. For example, within a single tissue or cell type, multiple mutational ‘mechanisms’ may lead to the development of cancer. As such, heterogeneity frequently exists between cancer cells taken from tumors of the same tissue and same type that have originated in different individuals. Frequently-observed mutational ‘mechanisms’ associated with some cancers may differ between one tissue type and another (e.g., frequently-observed mutational ‘mechanisms’ leading to colon cancer may differ from frequently-observed mechanisms leading to leukemias). It is therefore often difficult to predict whether a particular cancer will respond to a particular chemotherapeutic agent. (Cancer Medicine, 5th Edition, Bast et al. eds., B.C. Decker Inc., Hamilton, Ontario).

Surgery and radiotherapy may be curative if a cancer is found early, but current drug therapies for metastatic disease are mostly palliative and seldom offer a long-term cure. Even with the new chemotherapies entering the market, improvement in patient survival is measured in months rather than in years, and the need continues for new drugs effective both in combination with existing agents as first line therapy and as second and third line therapies in treatment of resistant tumors.

β-lapachone is an agent with a reported anti-cancer activity in a limited number of cancers. For example, there is reported a method and composition for the treatment of tumors, which comprises the administration of an effective amount of β-lapachone, in combination with a taxane derivative (U.S. Pat. No. 6,664,288; WO00/61142). Additionally, U.S. Pat. No. 6,245,807 discloses the use of β-lapachone, amongst other β-lapachone derivatives, for use in treatment of human prostate disease. As a single agent, β-lapachone has also been reported to decrease the number of tumors, reduce tumor size, or increase the survival time, or a combination of these, in xenotransplant mouse models of human ovarian cancer (Li, C. J. et al., (1999) Proc. Natl. Acad. Sci. USA, 96(23): 13369-13374), human prostate cancer (Li, C. J. et al., (1999) Proc. Natl. Acad. Sci. USA, 96(23): 13369-13374), human breast cancer (Li, C. J. et al., (2000) AACR Proc., p. 9), and human multiple myeloma (U.S. Patent Application Publication No. 2003-0036515; WO 03/011224).

### SUMMARY OF THE INVENTION

The present invention provides a method of treating cancer or a precancerous condition or preventing cancer in a subject, the method comprising administering to the subject a therapeutically effective amount of a pharmaceutical composition comprising β-lapachone, or a derivative or analog thereof, or pharmaceutically acceptable salt thereof, or a metabolite thereof, and a pharmaceutically acceptable carrier such that the composition maintains a plasma concentration of about 0.15 μM to about 50 μM and treats the cancer or precancerous condition or prevents the cancer.

The present invention also provides a method of treating cancer or a precancerous condition or preventing cancer in a subject, the method comprising administering to the subject a therapeutically effective amount of a pharmaceutical composition comprising β-lapachone, or a derivative or analog thereof, or pharmaceutically acceptable salt thereof, or a metabolite thereof, and a pharmaceutically acceptable carrier such that the composition maintains a plasma concentration of about 0.15 μM to about 50 μM, modulates one or more cell cycle checkpoints in one or more cancer cells and treats the cancer or precancerous condition or prevents the cancer.

The present invention also provides a method of treating cancer or a precancerous condition or preventing cancer in a subject, the method comprising administering to the subject a therapeutically effective amount of a pharmaceutical composition comprising β-lapachone, or a derivative or analog thereof, or pharmaceutically acceptable salt thereof, or a metabolite thereof, and a pharmaceutically acceptable carrier such that the composition maintains a plasma concentration of about 0.15 μM to about 50 μM, modulates cell death selectively in one or more cancer cells and treats the cancer or precancerous condition or prevents the cancer.

The present invention also provides a method of treating or preventing a cell proliferative disorder in a subject, the method comprising administering to the subject a therapeutically effective amount of a pharmaceutical composition comprising β-lapachone, or a derivative or analog thereof, or pharmaceutically acceptable salt thereof, or a metabolite thereof, and a pharmaceutically acceptable carrier such that the composition maintains a plasma concentration of about 0.15 μM to about 50 μM and treats or prevents the cell proliferative disorder.

The plasma concentration can be about 0.1 μM to about 100 μM, about 0.125 μM to about 75 μM; about 0.15 μM to about 50 μM; about 0.175 μM to about 30 μM; and about 0.2 μM to about 20 μM.

The subject can be exposed to the pharmaceutical composition in an AUC range of about 0.5 μM-hr to about 100 μM-hr, about 0.5 μM-hr to about 50 μM-hr, about 1 μM-hr to about 25 μm-hr, about 1 μM-hr to about 10 μM-hr; about 1.25 μM-hr to about 6.75 μM-hr, about 1.5 μM-hr to about 6.5 μM-hr.

In a preferred embodiment, the subject is a mammal. More preferably, the subject is a human.

The pharmaceutical composition can be administered at a dosage from about 2 mg/m² to 5000 mg/m² per day, more preferably from about 20 mg/m² to 2000 mg/m² per day, more preferably from about 20 mg/m² to 500 mg/m² per day, most preferably from about 30 to 300 mg/m² per day. Preferably, 2 mg/m² to 5000 mg/m² per day is the administered dosage for a human.

The pharmaceutical composition can be administered intravenously, orally or intraperitoneally.

The cancer can be multiple myeloma, chronic myelogenous leukemia, pancreatic cancer, non-small cell lung cancer, lung cancer, breast cancer, colon cancer, ovarian cancer, prostate cancer, malignant melanoma, non-melanoma skin cancers, hematologic tumors, hematologic tumors, hematologic malignancies, childhood leukemia, childhood lymphomas, multiple myeloma, Hodgkin's disease, lymphomas of lymphocytic origin, lymphomas of cutaneous origin, acute leukemia, chronic leukemia, acute lymphoblastic leukemia, acute myelocytic leukemia, chronic myelocytic leukemia, plasma cell neoplasm, lymphoid neoplasm, cancers associated with AIDS, cancers of the tongue, mouth, pharynx, and oral cavity, esophageal cancer, stomach cancer, cancer of the small intestine, anal cancer, cancer of the anal canal, anorectal cancer, liver cancer, intrahepatic bile duct cancer, gallbladder cancer, biliary cancer, cancer of other digestive organs, cancer of the larynx, bone and joint cancer, uterine cancer, cervical cancer, uterine corpus cancer, cancer of the vulva, vaginal cancer, testicular cancer, penile cancer, urinary bladder cancer, kidney cancer, renal cancer, cancer of the ureter and other urinary organs, ocular cancer, brain and nervous system cancer, CNS cancers, or thyroid cancer.

The pharmaceutically acceptable carrier can be a solubilizing carrier molecule. Preferably, the solubilizing carrier molecule can be Poloxamer, Povidone K17, Povidone K12, Tween 80, ethanol, Cremophor/ethanol, Lipiodol, polyethylene glycol (PEG) 400, propylene glycol, Trappsol, alpha-cyclodextrin or analogs thereof, beta-cyclodextrin or analogs thereof, and gamma-cyclodextrin or analogs thereof.

In other embodiments, treatment can include a reduction in tumor size, reduction in tumor number, decrease in tumor growth rate, decrease of tumor regrowth, increase in average survival time of a population of treated subjects in comparison to an untreated population, or an increase in average survival time of a population of treated subjects in comparison to a population receiving monotherapy with a drug that is not β-lapachone.

The activation of one or more cell cycle checkpoints can activate one or more cell cycle pathways or cell cycle regulators in one or more cancer cells.

Administration of the pharmaceutical composition of the invention can activate a cell cycle checkpoint, activate E2F transcription factor pathway, induce elevation of an E2F transcription factor, stimulate unscheduled activation of an E2F transcription factor or induce cell death selectively. Preferably, the cell cycle checkpoint that is activated is a G1 or S cell cycle checkpoint, elevation of an E2F transcription factor is selective, activation of an E2F transcription factor is selective and the cell death is apoptosis, necrosis or senescence.

In a preferred embodiment, the therapeutically effective amount is not cytotoxic to normal cells and does not affect normal cell viability.

The present invention can also include administering a therapeutically effective amount of a second anti-cancer agent or a second anti-proliferative agent, or a derivative or analog thereof along with the pharmaceutical composition comprising β-lapachone, or a derivative or analog thereof, or pharmaceutically acceptable salt thereof, or a metabolite thereof, and a pharmaceutically acceptable carrier.

The second anti-cancer agent or anti-proliferative agent can be paclitaxel (Taxol®), docetaxel, vincristin, vinblastin, nocodazole, epothilones, navelbine, etoposide, adriamycin, camptothecin, daunorubicin, dactinomycin, mitoxantrone, amsacrine, epirubicin, idarubicin, gemcitabine and imatinib.

The pharmaceutical composition of the present invention can be administered simultaneously with or following administration of the second anti-cancer agent or second anti-proliferative agent, more preferably the second anti-cancer agent or second anti-proliferative agent is administered following administration of the pharmaceutical composition, most preferably the second anti-cancer agent or second anti-proliferative agent is administered within 24 hours after the pharmaceutical composition is administered.

Other features and advantages of the invention will be apparent from the following detailed description and claims.

### BRIEF DESCRIPTION OF THE FIGURES

FIG. 1 is a line graph which shows the plasma concentration of β-lapachone in tumor-bearing female nude (Ncr) mice following the IP administration of 150 mg/m² of β-lapachone.

FIG. 2 is a line graph which shows the plasma concentration of β-lapachone in tumor-bearing female nude (Ncr) mice following the IP administration of 50 mg/kg or 10 mg/kg of β-lapachone in the HPBCD formulation.

FIG. 3 is a line graph which shows the pharmacokinetics of β-lapachone administered to rats as a one-hour or ten-minute intravenous infusion in the HPBCD formulation.

FIG. 4 is a line graph which shows the pharmacokinetics of β-lapachone administered to dogs as a one-hour intravenous infusion in the HPBCD formulation.

FIG. 5 is a bar graph which shows effect of β-Lapachone on survival of human cancer cell lines in the NCI60 assay in vitro.

FIG. 6 is a bar graph which shows effect of β-Lapachone on survival of human colon cancer cell lines in the NCI60 assay in vitro.

FIG. 7 is a line graph which shows the effect of β-Lapachone on the growth of xenografted HT-29 human colon tumors in an athymic nude mouse model.

FIG. 8 is a bar graph which shows effect of β-Lapachone on survival of human lung cancer cell lines in the NCI60 assay in vitro.

FIG. 9 is a line graph which shows the effect of β-Lapachone on the growth of xenografted A549 human lung tumors in an athymic nude mouse model.

FIG. 10 is a photograph of a Western blot showing that E2F-1 protein expression is upregulated by β-Lapachone in human pancreatic cancer cells (Paca-2).

FIG. 11 is a bar graph which shows A) treatment with 25 and 50 mg/kg β-lapachone inhibited prostate tumor growth in a dose-dependent manner and B) greater suppression of prostate tumor growth when 100 mg/kg β-lapachone was administered to established tumors.

FIG. 12 is a series of line and bar graphs which show the differential effects of β-Lapachone on human multiple myeloma (MM) cells vs. normal human Peripheral Blood Mononuclear Cells (PBMC).

FIG. 13 is a photograph of a colony formation assay showing the differential effects of β-lapachone on human breast cancer cells (MCF-7) vs. normal human breast epithelial cells (MCF-10A).

### DETAILED DESCRIPTION OF THE INVENTION

The present invention provides a method of treating cancer or a precancerous condition or preventing cancer in a subject, the method comprising administering to the subject a therapeutically effective amount of a pharmaceutical composition comprising β-lapachone, or a derivative or analog thereof, or pharmaceutically acceptable salt thereof, or a metabolite thereof, and a pharmaceutically acceptable carrier such that the composition maintains a plasma concentration of about 0.15 μM to about 50 μM and treats the cancer or precancerous condition or prevents the cancer.

The present invention also provides a method of treating cancer or a precancerous condition or preventing cancer in a subject, the method comprising administering to the subject a therapeutically effective amount of a pharmaceutical composition comprising β-lapachone, or a derivative or analog thereof, or pharmaceutically acceptable salt thereof, or a metabolite thereof, and a pharmaceutically acceptable carrier such that the composition maintains a plasma concentration of about 0.15 μM to about 50 μM, modulates one or more cell cycle checkpoints in one or more cancer cells and treats the cancer or precancerous condition or prevents the cancer.

The present invention also provides a method of treating cancer or a precancerous condition or preventing cancer in a subject, the method comprising administering to the subject a therapeutically effective amount of a pharmaceutical composition comprising β-lapachone, or a derivative or analog thereof, or pharmaceutically acceptable salt thereof, or a metabolite thereof, and a pharmaceutically acceptable carrier such that the composition maintains a plasma concentration of about 0.15 μM to about 50 μM, modulates cell death selectively in one or more cancer cells and treats the cancer or precancerous condition or prevents the cancer.

The present invention also provides a method of treating or preventing a cell proliferative disorder in a subject, the method comprising administering to the subject a therapeutically effective amount of a pharmaceutical composition comprising β-lapachone, or a derivative or analog thereof, or pharmaceutically acceptable salt thereof, or a metabolite thereof, and a pharmaceutically acceptable carrier such that the composition maintains a plasma concentration of about 0.15 μM to about 50 μM and treats or prevents the cell proliferative disorder.

The present invention also provides the use of β-lapachone for the preparation of a medicament useful for the treatment of cancer. The invention also provides the use of β-lapachone for the preparation of a medicament useful for the treatment or prevention of a cell proliferative disorder.

β-lapachone (3,4-dihydro-2,2-dimethyl-2H-naphtho [1,2-b]pyran-5,6-dione), also referred to as CO-501 and ARQ-501 herein, is a simple non-water soluble orthonapthoquinone, was first isolated in 1882 by Paterno from the heartwood of the lapacho tree (See Hooker, S C, (1936) I. Am. Chem. Soc. 58:1181-1190; Goncalves de Lima, 0, et al., (1962) Rev. Inst. Antibiot. Univ. Recife. 4:3-17). The structure of β-Lapachone was established by Hooker in 1896 and it was first synthesized by Fieser in 1927 (Hooker, S C, (1936) I. Am. Chem. Soc. 58:1181-1190). β-lapachone can, for example, be obtained by simple sulfuric acid treatment of the naturally occurring lapachol, which is readily isolated from Tabebuia avellenedae growing mainly in Brazil, or is easily synthesized from seeds of lomatia growing in Australia (Li, C J, et al., (1993) J. Biol. Chem. 268:22463-33464). Methods for formulating β-Lapachone can be accomplished as described in U.S. Pat. No. 6,458,974 and U.S. Publication No. US-2003-0091639-A1.

As used herein, derivatives or analogs of β-Lapachone include, for example, 3,4-dihydro-2,2-dimethyl-3-(3-methyl-2-butenyl)-2H-naphtho[1,2-b]pyran-5,6-dione, 3,4-dihydro-2,2-dimethyl-2H-naphtho[1,2-b]thiopyran-5,6-dione and 3,4-dihydro-4,4-dimethyl-2H-naphtho[1,2-b]thiopyran-5,6-dione. Other derivatives or analogs of β-lapachone are described in PCT International Application PCT/US93/07878 (WO94/04145), and U.S. Pat. No. 6,245,807. PCT International Application PCT/US00/10169 (WO 00/61142), discloses β-lapachone, which may have a variety of substituents at the 3-position as well as in place of the methyl groups attached at the 2-position. U.S. Pat. Nos. 5,763,625, 5,824,700, and 5,969,163, disclose analogs and derivatives with a variety of substituents at the 2-, 3- and 4-positions. Furthermore, a number of journals report β-lapachone analogs and derivatives with substituents at one or more of the following positions: 2-, 3-, 8- and/or 9-positions, (See, Sabba et al., (1984) J Med Chem 27:990-994 (substituents at the 2-, 8- and 9-positions); (Portela and Stoppani, (1996) Biochem Pharm 51:275-283 (substituents at the 2- and 9-positions); Goncalves et al., (1998) Molecular and Biochemical Parasitology 1: 167-176 (substituents at the 2- and 3-positions)). Other derivatives or analogs of β-lapachone have sulfur-containing hetero-rings in the “α” and “β” positions of lapachone (Kurokawa S, (1970) Bulletin of The Chemical Society of Japan 43:1454-1459; Tapia, R A et al., (2000) Heterocycles 53(3):585-598; Tapia, R A et al., (1997) Tetrahedron Letters 38(1):153-154; Chuang, C P et al., (1996) Heterocycles 40(10):2215-2221; Suginome H et al., (1993) Journal of the Chemical Society, Chemical Communications 9:807-809; Tonholo J et al., (1988) Journal of the Brazilian Chemical Society 9(2):163-169; and Krapcho A P et al., (1990) Journal of Medicinal Chemistry 33(9):2651-2655).

All stereoisomers of the compounds of the instant invention are contemplated, either in admixture or in pure or substantially pure form. The definition of the compounds according to the invention embraces all possible stereoisomers (e.g., the R and S configurations for each asymmetric center) and their mixtures. It very particularly embraces the racemic forms and the isolated optical isomers having a specified activity. The racemic forms can be resolved by physical methods, such as, for example, fractional crystallization, separation or crystallization of diastereomeric derivatives or separation by chiral column chromatography. The individual optical isomers can be obtained from the racemates by conventional methods, such as, for example, salt formation with an optically active acid followed by crystallization. Furthermore, all geometric isomers, such as E- and Z-configurations at a double bond, are within the scope of the invention unless otherwise stated. Certain compounds of this invention may exist in tautomeric forms. All such tautomeric forms of the compounds are considered to be within the scope of this invention unless otherwise stated.

As used herein, the term “salt” is a pharmaceutically acceptable salt and can include acid addition salts including hydrochlorides, hydrobromides, phosphates, sulphates, hydrogen sulphates, alkylsulphonates, arylsulphonates, acetates, benzoates, citrates, maleates, fumarates, succinates, lactates, and tartrates; alkali metal cations such as Na, K, Li, alkali earth metal salts such as Mg or Ca, or organic amine salts.

As used herein, the term “metabolite” means a product of metabolism of β-lapachone, or a pharmaceutically acceptable salt thereof that exhibits a similar activity in vivo to β-lapachone.

As used herein, the “subject” can be any mammal, e.g., a human, a primate, mouse, rat, dog, cat, cow, horse, pig, sheep, goat, chicken, camel, bison. In a preferred aspect, the subject is a human in need thereof.

As used herein, the term “cell proliferative disorder” refers to conditions in which unregulated or abnormal growth, or both, of cells can lead to the development of an unwanted condition or disease, which may or may not be cancerous. In one aspect, a cell proliferative disorder includes, for example, skin cancer and precancerous conditions of the skin. A “cell proliferative disorder of the skin” is a cell proliferative disorder involving cells of the skin. In one aspect, a cell proliferative disorder includes a pre-cancer. In another aspect, a cell proliferative disorder includes hyperplasia, metaplasia, and dysplasia.

As used herein, a “normal cell” is a cell that cannot be classified as part of a “cell proliferative disorder.” In one aspect, a normal cell lacks unregulated or abnormal growth, or both, that can lead to the development of an unwanted condition or disease. Preferably, a normal cell possesses normally functioning cell cycle checkpoint control mechanisms.

As used herein, “contacting a cell” refers to a condition in which a compound or other composition of matter is in direct contact with a cell, or is close enough to induce a desired biological effect in a cell.

As used herein, “monotherapy” refers to administration of a single active or therapeutic compound to a subject in need thereof. Preferably, monotherapy will involve administration of a therapeutically effective amount of an active compound. For example, β-lapachone monotherapy for cancer comprises administration of a therapeutically effective amount of β-lapachone, or a pharmaceutically acceptable salt, or metabolite thereof, to a subject in need of treatment of cancer. Monotherapy may be contrasted with combination therapy, in which a combination of multiple active compounds is administered, preferably with each component of the combination present in a therapeutically effective amount. In one aspect, β-lapachone monotherapy is more effective than combination therapy in inducing a desired biological effect.

In one aspect, combination therapy includes β-lapachone with Taxol®; β-lapachone with docetaxel; β-lapachone with vincristin; β-lapachone with vinblastin; β-lapachone with nocodazole; β-lapachone with teniposide; β-lapachone with etoposide; β-lapachone with adriamycin; β-lapachone with epothilone; β-lapachone with navelbine; β-lapachone with camptothecin; β-lapachone with daunorubicin; β-lapachone with dactinomycin; β-lapachone with mitoxantrone; β-lapachone with amsacrine; β-lapachone with epirubicin; β-lapachone with idarubicin; β-lapachone with gemcitabine or β-lapachone with imatinib. In another aspect, combination therapy includes reduced β-lapachone with Taxol®; reduced β-lapachone with docetaxel; reduced β-lapachone with vincristin; reduced β-lapachone with vinblastin; reduced β-lapachone with nocodazole; reduced β-lapachone with teniposide; reduced β-lapachone with etoposide; reduced β-lapachone with adriamycin; reduced β-lapachone with epothilone; reduced β-lapachone with navelbine; reduced β-lapachone with camptothecin; reduced β-lapachone with daunorubicin; reduced β-lapachone with dactinomycin; reduced β-lapachone with mitoxantrone; reduced β-lapachone with amsacrine; reduced β-lapachone with epirubicin; reduced β-lapachone with idarubicin; reduced β-lapachone with gemcitabine or reduced β-lapachone with imatinib.

In a preferred aspect of the invention, the cell proliferation disorder is cancer. Cancer includes multiple myeloma, chronic myelogenous leukemia, pancreatic cancer, non-small cell lung cancer, lung cancer, breast cancer, colon cancer, ovarian cancer, prostate cancer, malignant melanoma, non-melanoma skin cancers, hematologic tumors, hematologic tumors, hematologic malignancies, childhood leukemia, childhood lymphomas, multiple myeloma, Hodgkin's disease, lymphomas of lymphocytic origin, lymphomas of cutaneous origin, acute leukemia, chronic leukemia, acute lymphoblastic leukemia, acute myelocytic leukemia, chronic myelocytic leukemia, plasma cell neoplasm, lymphoid neoplasm, cancers associated with AIDS, cancers of the tongue, mouth, pharynx, and oral cavity, esophageal cancer, stomach cancer, cancer of the small intestine, anal cancer, cancer of the anal canal, anorectal cancer, liver cancer, intrahepatic bile duct cancer, gallbladder cancer, biliary cancer, cancer of other digestive organs, cancer of the larynx, bone and joint cancer, uterine cancer, cervical cancer, uterine corpus cancer, cancer of the vulva, vaginal cancer, testicular cancer, penile cancer, urinary bladder cancer, kidney cancer, renal cancer, cancer of the ureter and other urinary organs, ocular cancer, brain and nervous system cancer, CNS cancers, and thyroid cancer.

In a preferred aspect, compositions of the present invention may be used to treat colon cancer or cell proliferative disorders of the colon. In one aspect, colon cancer includes all forms of cancer of the colon. In another aspect, colon cancer includes sporadic and hereditary colon cancers. In another aspect, colon cancer includes malignant colon neoplasms, carcinoma in situ, typical carcinoid tumors, and atypical carcinoid tumors. In another aspect, colon cancer includes adenocarcinoma, squamous cell carcinoma, and adenosquamous cell carcinoma. In another aspect, colon cancer includes stage I, stage II, stage III, or stage IV colon cancer. In another aspect, colon cancer is associated with a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis. In another aspect, colon cancer is caused by a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis.

In one aspect, cell proliferative disorders of the colon include all forms of cell proliferative disorders affecting colon cells. In one aspect, cell proliferative disorders of the colon include colon cancer, precancerous conditions of the colon, adenomatous polyps of the colon and metachronous lesions of the colon. In one aspect, a cell proliferative disorder of the colon includes adenoma. In one aspect, cell proliferative disorders of the colon are characterized by hyperplasia, metaplasia, and dysplasia of the colon. In another aspect, prior colon diseases that may predispose individuals to development of cell proliferative disorders of the colon include prior colon cancer. In another aspect, current disease that may predispose individuals to development of cell proliferative disorders of the colon include Crohn's disease and ulcerative colitis. In one aspect, a cell proliferative disorder of the colon is associated with a mutation in a gene selected from the group consisting of p53, ras, FAP and DCC. In another aspect, an individual has an elevated risk of developing a cell proliferative disorder of the colon due to the presence of a mutation in a gene selected from the group consisting of p53, ras, FAP and DCC.

In a preferred aspect, compositions of the present invention may be used to treat lung cancer or cell proliferative disorders of the lung. In one aspect, lung cancer includes all forms of cancer of the lung. In another aspect, lung cancer includes malignant lung neoplasms, carcinoma in situ, typical carcinoid tumors, and atypical carcinoid tumors. In another aspect, lung cancer includes small cell lung cancer (“SCLC”), non-small cell lung cancer (“NSCLC”), squamous cell carcinoma, adenocarcinoma, small cell carcinoma, large cell carcinoma, adenosquamous cell carcinoma, and mesothelioma. In another aspect, lung cancer includes “scar carcinoma,” bronchioalveolar carcinoma, giant cell carcinoma, spindle cell carcinoma, and large cell neuroendocrine carcinoma. In another aspect, lung cancer includes lung neoplasms having histologic and ultrastructual heterogeneity (e.g., mixed cell types).

In one aspect, cell proliferative disorders of the lung include all forms of cell proliferative disorders affecting lung cells. In one aspect, cell proliferative disorders of the lung include lung cancer, precancerous conditions of the lung. In one aspect, cell proliferative disorders of the lung include hyperplasia, metaplasia, and dysplasia of the lung. In another aspect, cell proliferative disorders of the lung include asbestos-induced hyperplasia, squamous metaplasia, and benign reactive mesothelial metaplasia. In another aspect, cell proliferative disorders of the lung include replacement of columnar epithelium with stratified squamous epithelium, and mucosal dysplasia. In another aspect, individuals exposed to inhaled injurious environmental agents such as cigarette smoke and asbestos may be at increased risk for developing cell proliferative disorders of the lung. In another aspect, prior lung diseases that may predispose individuals to development of cell proliferative disorders of the lung include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, and Hodgkin's disease.

In a preferred aspect, compositions of the present invention may be used to treat pancreatic cancer or cell proliferative disorders of the pancreas. In one aspect, pancreatic cancer includes all forms of cancer of the pancreas. In another aspect, pancreatic cancer includes ductal adenocarcinoma. In another aspect, pancreatic cancer includes adenosquamous carcinoma, pleomorphic giant cell carcinoma, mucinous adenocarcinoma, and osteoclast-like giant cell carcinoma. In another aspect, pancreatic cancer includes mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma. In another aspect, pancreatic cancer includes pancreatic neoplasms having histologic and ultrastructual heterogeneity (e.g., mixed cell types).

In one aspect, cell proliferative disorders of the pancreas include all forms of cell proliferative disorders affecting pancreatic cells. In one aspect, cell proliferative disorders of the pancreas include pancreatic cancer, precancerous conditions of the pancreas, hyperplasia of the pancreas, and dysaplasia of the pancreas. In another aspect, prior pancreatic diseases may predispose indivduals to the development of cell proliferative disorders of the pancreas. In another aspect, existing pancreatic diseases that may predispose individuals to development of cell proliferative disorders of the pancreas include diabetes mellitus and pancreatitis.

In a preferred aspect, compositions of the present invention may be used to treat a cancer selected from the group consisting of a hematologic cancer of the present invention or a hematologic cell proliferative disorder of the present invention. In one aspect, a cancer selected from the group consisting of a hematologic cancer of the present invention includes lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, and mast cell leukemia), myeloid neoplasms and mast cell neoplasms. As used herein, the term “leukemia” does not encompass multiple myeloma.

In one aspect, a hematologic cell proliferative disorder of the present invention includes lymphoma, leukemia, myeloid neoplasms, mast cell neoplams, myelodysplasia, benign monoclonal gammopathy, lymphomatoid granulomatosis, lymphomatoid papulosis, polycythemia vera, chronic myelocytic leukemia, agnogenic myeloid metaplasia, and essential thrombocythemia. In another aspect, a hematologic cell proliferative disorder of the present invention includes hyperplasia, dysplasia, and metaplasia.

As used herein, “therapeutically effective amount” means an amount of a drug or pharmaceutical agent that will elicit a desired biological or medical response of a tissue, system, animal or human that is being sought by a researcher or clinician. In a preferred aspect, the biological or medical response is treatment of a cancer of the present invention. In another aspect, the biological or medical response is treatment or prevention of a cell proliferative disorder of a cancer or pre-cancer of the present invention.

In one aspect, the plasma concentration can be about 0.1 μM to about 100 μM, about 0.125 μM to about 75 μM; about 0.15 μM to about 50 μM; about 0.175 μM to about 30 μM; and about 0.2 μM to about 20 μM.

In another aspect, the pharmaceutical composition can maintain a suitable plasma concentration for at least a month, at least a week, at least 24 hours, at least 12 hrs, at least 6 hrs, at least 1 hour. In a further aspect, a suitable plasma concentration of the pharmaceutical composition can be maintained indefinitely.

In yet another aspect, the subject can be exposed to the pharmaceutical composition in a AUC range of about 0.5 μM-hr to about 100 μM-hr, about 0.5 μM-hr to about 50 μm-hr, about 1 μM-hr to about 25 μM-hr, about 1 μm-hr to about 10 μM-hr; about 1.25 μM-hr to about 6.75 μM-hr, about 1.5 μM-hr to about 6.5 μM-hr.

In one aspect, treating a cancer of the present invention results in a reduction in size of a tumor. A reduction in size of a tumor may also be referred to as “tumor regression.” Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Size of a tumor may be measured by any reproducible means of measurement. In a preferred aspect, size of a tumor may be measured as a diameter of the tumor.

In another aspect, treating a cancer of the present invention results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. In a preferred aspect, number of tumors may be measured by counting tumors visible to the naked eye, or at a specified magnification. In a preferred aspect, the specified magnification is 2×, 3×, 4×, 5×, 10×, or 50×.

In another aspect, treating a cancer of the present invention results in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. In a preferred aspect, an increase in average survival time of a population may be measured, for example, by calculating for a population the average length of survival following initiation of treatment with an active compound. In an another preferred aspect, an increase in average survival time of a population may also be measured, for example, by calculating for a population the average length of survival following completion of a first round of treatment with an active compound.

In another aspect, treating a cancer of the present invention results in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. In a preferred aspect, an increase in average survival time of a population may be measured, for example, by calculating for a population the average length of survival following initiation of treatment with an active compound. In an another preferred aspect, an increase in average survival time of a population may also be measured, for example, by calculating for a population the average length of survival following completion of a first round of treatment with an active compound.

In another aspect, treating a cancer of the present invention results in increase in average survival time of a population of treated subjects in comparison to a population receiving monotherapy with a drug that is not β-lapachone, or a pharmaceutically acceptable salt, or metabolite thereof. Preferably, the average survival time is increased by more than 30 days; more preferably, by more than 60 days; more preferably, by more than 90 days; and most preferably, by more than 120 days. An increase in average survival time of a population may be measured by any reproducible means. In a preferred aspect, an increase in average survival time of a population may be measured, for example, by calculating for a population the average length of survival following initiation of treatment with an active compound. In an another preferred aspect, an increase in average survival time of a population may also be measured, for example, by calculating for a population the average length of survival following completion of a first round of treatment with an active compound.

In another aspect, treating a cancer of the present invention results in a decrease in the mortality rate of a population of treated subjects in comparison to a population receiving carrier alone. In another aspect, treating a cancer of the present invention results in a decrease in the mortality rate of a population of treated subjects in comparison to an untreated population. In a further aspect, treating a cancer of the present invention results a decrease in the mortality rate of a population of treated subjects in comparison to a population receiving monotherapy with a drug that is not β-lapachone, or a pharmaceutically acceptable salt, or metabolite thereof. Preferably, the mortality rate is decreased by more than 2%; more preferably, by more than 5%; more preferably, by more than 10%; and most preferably, by more than 25%. In a preferred aspect, a decrease in the mortality rate of a population of a population may be measured by any reproducible means. In another preferred aspect, a decrease in the mortality rate of a population may be measured, for example, by calculating for a population the average number of disease-related deaths per unit time following initiation of treatment with an active compound. In another preferred aspect, a decrease in the mortality rate of a population may also be measured, for example, by calculating for a population the average number of disease-related deaths per unit time following completion of a first round of treatment with an active compound.

In another aspect, treating a cancer of the present invention results in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; even more preferably, reduced by at least 50%; and most preferably, reduced by at least 75%. Tumor growth rate may be measured by any reproducible means of measurement. In a preferred aspect, tumor growth rate is measured according to a change in tumor diameter per unit time.

In another aspect, treating a cancer of the present invention results in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. In a preferred aspect, tumor regrowth is measured, for example, by measuring an increase in the diameter of a tumor after a prior tumor shrinkage that followed treatment. In another preferred aspect, a decrease in tumor regrowth is indicated by failure of tumors to reoccur after treatment has stopped.

In another aspect, treating or preventing a cell proliferative disorder of a cancer or pre-cancer of the present invention results in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; even more preferably, by at least 50%; and most preferably, by at least 75%. The rate of cellular proliferation may be measured by any reproducible means of measurement. In a preferred aspect, the rate of cellular proliferation is measured, for example, by measuring the number of dividing cells in a tissue sample per unit time.

In another aspect, treating or preventing a cell proliferative disorder of the cancer or pre-cancer of the present invention results in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. In a preferred aspect, the proportion of proliferating cells is measured, for example, by quantifying the number of dividing cells relative to the number of nondividing cells in a tissue sample. In another preferred aspect, the proportion of proliferating cells is equivalent to the mitotic index.

In another aspect, treating or preventing a cell proliferative disorder of a cancer or precancer of the present invention results in a decrease in size of an area or zone of cellular proliferation. Preferably, after treatment, size of an area or zone of cellular proliferation is reduced by at least 5% relative to its size prior to treatment; more preferably, reduced by 10%; more preferably, reduced by at least 20%; more preferably, reduced by at least 30%; more preferably, reduced by at least 40%; even more preferably, reduced by at least 50%; and most preferably, reduced by greater than 75%. Size of an area or zone of cellular proliferation may be measured by any reproducible means of measurement. In a preferred aspect, size of an area or zone of cellular proliferation may be measured as a diameter of an area or zone of cellular proliferation.

As used herein, the term “selectively” means tending to occur at a higher frequency in one population than in another population. In one aspect, the compared populations are cell populations. In a preferred aspect, β-lapachone, or a pharmaceutically acceptable salt, or metabolite thereof, acts selectively on a cancer or precancer cell but not on a normal cell. Preferably, an event occurs selectively in population A relative to population B if it occurs greater than two times more frequently in population A as compared to population B. More preferably, an event occurs selectively if it occurs greater than five times more frequently in population A. More preferably, an event occurs selectively if it occurs greater than ten times more frequently in population A; more preferably, greater than fifty times; even more preferably, greater than 100 times; and most preferably, greater than 1000 times more frequently in population A as compared to population B. For example, cell death would be said to occur selectively in cancer cells if it occurred greater than twice as frequently in cancer cells as compared to normal cells.

In one aspect, treating a cancer or pre-cancer of the present invention or a cell proliferative disorder results in cell death and preferably cell death results in a decrease of at least 10% of the cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. In one aspect, number of cells in a population is measured by fluorescence activated cell sorting (FACS). In another aspect, number of cells in a population is measured by immunofluorescence microscopy. In another aspect, number of cells in a population is measured by light microscopy. In another aspect, methods of measuring cell death are as shown in Li et al., (2003) Proc Natl Acad Sci USA. 100(5): 2674-8. In a preferred aspect, cell death results from apoptosis, necrosis or senescence.

In a preferred aspect, an effective amount of β-lapachone, or a pharmaceutically acceptable salt, or metabolite thereof is not cytotoxic to normal cells. A therapeutically effective amount of a compound is not cytotoxic to normal cells if administration of the compound at a therapeutically effective amount does not induce apoptosis in greater than 10% of normal cells. A therapeutically effective amount of a compound does not affect the viability of normal cells if administration of the compound at a therapeutically effective amount does not induce cell death in greater than 10% of normal cells.

While not limited by theory, the present invention includes and is based in part on an understanding of, and methods for, the activation of cell cycle checkpoints by β-lapachone. The activation of cell cycle checkpoints in general is referred to as Activated Checkpoint Therapy™, or ACT™. The fact that β-lapachone is an effective checkpoint activator facilitates its broad applicability to a range of cancers and pre-cancers (WO 04/007531).

Briefly, many cancer cells are defective in their cell cycle checkpoint functions secondary to mutations in one of their molecular modulators, e.g., p53. It is in part, for this reason, that cancer cells have accumulated genetic errors during the carcinogenic process. Therapeutic agents that activate cell cycle checkpoint functions can selectively promote cell death in cancer cells, since cell death appears to be induced by the conflict between the uncontrolled-proliferation drive in cancer cells and the checkpoint delays induced artificially. ACT™ takes advantage of the tendency of cell death to occur at checkpoints during the cell proliferation cycle by activating one or more checkpoints, thereby producing conflicting signals regarding cell cycle progression versus arrest. If more than one checkpoint is activated, cancer cells with uncontrolled proliferation signals and genetic abnormalities are blocked at multiple checkpoints, creating “collisions” that promote synergistic cell death.

ACT™ offers selectivity against cancer cells as compared to normal cells and is therefore safer than less selective therapies. First, the ACT™ method modulates (activates or inhibits) but does not disrupt cell cycle checkpoints. Second, normal cells with well-controlled proliferation signals can be delayed at checkpoints in a regulated fashion, resulting in no cell death-prone collisions. Third, normal cells with intact G1 checkpoint control are expected to arrest in G1. Cancer cells, on the other hand, are expected to be delayed in S-, G2-, and M-phases, since most cancer cells harbor G1 checkpoint defects, making cancer cells more sensitive to drugs imposing S and M phase checkpoints. β-lapachone is a G1 and S phase compound, and contacting a cell with β-lapachone results in activation of a G1 or S cell cycle checkpoint.

In one aspect, activating refers to placing one or more compositions of matter (e.g., protein or nucleic acid) in a state suitable for carrying out a desired biological function. In one aspect, a composition of matter capable of being activated also has an unactivated state. In one aspect, an activated composition of matter may have an inhibitory or stimulatory biological function, or both.

In one aspect, elevation refers to an increase in a desired biological activity of a composition of matter (e.g., a protein or a nucleic acid). In one aspect, elevation may occur through an increase in concentration of a composition of matter.

As used herein, “a cell cycle checkpoint pathway” refers to a biochemical pathway that is involved in modulation of a cell cycle checkpoint. A cell cycle checkpoint pathway may have stimulatory or inhibitory effects, or both, on one or more functions comprising a cell cycle checkpoint. A cell cycle checkpoint pathway is comprised of at least two compositions of matter, preferably proteins, both of which contribute to modulation of a cell cycle checkpoint. A cell cycle checkpoint pathway may be activated through an activation of one or more members of the cell cycle checkpoint pathway. Preferably, a cell cycle checkpoint pathway is a biochemical signaling pathway.

As used herein, “cell cycle checkpoint regulator” refers to a composition of matter that can function, at least in part, in modulation of a cell cycle checkpoint. A cell cycle checkpoint regulator may have stimulatory or inhibitory effects, or both, on one or more functions comprising a cell cycle checkpoint. In one aspect, a cell cycle checkpoint regulator is a protein. In another aspect, a cell cycle checkpoint regulator is a non-protein.

In one aspect, stimulation of unscheduled expression of a checkpoint molecule by β-lapachone, or a pharmaceutically acceptable salt, or metabolite thereof, selectively triggers cell death in cells with defective checkpoints, a hallmark of cancer and pre-cancer cells. In one aspect, contacting a cell with β-lapachone stimulates unscheduled expression of the checkpoint molecule E2F. As used herein, “E2F” is the E2F transcription factor family (including but not limited to E2F-1, E2F-2, E2F-3).

In normal cells with their intact regulatory mechanisms, imposed expression of a checkpoint molecule (e.g., as induced by contacting a cell with β-lapachone, or a pharmaceutically acceptable salt, metabolite, analog or derivative thereof) results in an expression pattern that is not reported to be of substantial consequence. In contrast, cancer and pre-cancer cells have defective mechanisms, which result in unchecked or persistent expression, or both, of unscheduled checkpoint molecules, e.g. E2F, leading to selective cell death in cancer and pre-cancer cells. The present invention includes and provides for the unchecked or persistent expression, or both, of unscheduled checkpoint molecules by the administration of β-lapachone, or a pharmaceutically acceptable salt, or metabolite thereof.

In one aspect, contacting a cell with β-lapachone, or a pharmaceutically acceptable salt, or metabolite thereof results in activation of one or more cell cycle checkpoints. Preferably, administering to a patient in need thereof β-lapachone, or a pharmaceutically acceptable salt, or metabolite thereof results in activation of one or more cell cycle checkpoints.

In one aspect, contacting a cell with β-lapachone, or a pharmaceutically acceptable salt, or metabolite thereof results in activation of one or more cell cycle checkpoint regulators. Preferably, administering to a patient in need thereof β-lapachone, or a pharmaceutically acceptable salt, or metabolite thereof results in activation of one or more cell cycle checkpoint regulators.

In one aspect, contacting a cell with β-lapachone, or a pharmaceutically acceptable salt, or metabolite thereof modulates (induces or activates) cell death selectively in cells of a cancer of the present invention. Preferably, administering to a patient in need thereof β-lapachone, or a pharmaceutically acceptable salt, or metabolite thereof induces or activates cell death selectively in a cancer of the present invention cells. In another aspect, contacting a cell with β-lapachone, or a pharmaceutically acceptable salt, or metabolite thereof, induces cell death selectively in one or more cells affected by a cell proliferative disorder of a cancer or pre-cancer of the present invention. Preferably, administering to a subject in need thereof β-lapachone, or a pharmaceutically acceptable salt, or metabolite thereof, induces cell death selectively in one or more cells affected by a cell proliferative disorder of a cancer or pre-cancer of the present invention.

In one aspect, contacting a cell with β-lapachone, or a pharmaceutically acceptable salt, or metabolite thereof, results in activation of an E2F transcription factor pathway. Preferably, administering to a subject in need thereof β-lapachone, or a pharmaceutically acceptable salt, or metabolite thereof, results in activation of an E2F transcription factor pathway. In a preferred aspect, E2F activity is increased by more than 5%; more preferably, by more than 10%; more preferably, by more than 25%; more preferably, by more than 50%; and most preferably, by more than 2-fold. Methods of measuring induction of E2F activity and elevation of E2F levels are as shown in Li et al., (2003) Proc Natl Acad Sci USA. 100(5): 2674-8.

In one aspect, contacting a cell with β-lapachone, or a pharmaceutically acceptable salt, or metabolite thereof, results in elevation of an E2F transcription factor. Preferably, administering to a subject in need thereof β-lapachone, or a pharmaceutically acceptable salt, or metabolite thereof, results in elevation of an E2F transcription factor.

In one aspect, contacting a cell with β-lapachone, or a pharmaceutically acceptable salt, or metabolite thereof, results in elevation of an E2F transcription factor selectively in cancer or pre-cancer cells of the present invention but not in normal cells. Preferably, administering to a subject in need thereof β-lapachone, or a pharmaceutically acceptable salt, or metabolite thereof, results in elevation of an E2F transcription factor selectively in cancer or pre-cancer of the present invention cells but not in normal cells.

In one aspect, contacting a cell with β-lapachone, or a pharmaceutically acceptable salt, or metabolite thereof, stimulates unscheduled activation of an E2F transcription factor. Preferably, administering to a subject in need thereof β-lapachone, or a pharmaceutically acceptable salt, or metabolite thereof, stimulates unscheduled activation of an E2F transcription factor.

In one aspect, contacting a cell with β-lapachone, or a pharmaceutically acceptable salt, or metabolite thereof, stimulates unscheduled activation of an E2F transcription factor selectively in cancer or pre-cancer cells but not in normal cells. Preferably, administering to a subject in need thereof β-lapachone, or a pharmaceutically acceptable salt, or metabolite thereof, stimulates unscheduled activation of an E2F transcription factor selectively in cancer or pre-cancer cells but not in normal cells.

In a preferred aspect, the present invention relates to a method of treating or preventing cancer by administering a β-lapachone, or a pharmaceutically acceptable salt, or metabolite thereof to a subject in need thereof, where administration of the β-lapachone, or a pharmaceutically acceptable salt, or metabolite thereof results in one or more of the following: accumulation of cells in G1 and/or S phase of the cell cycle, cytotoxicity via cell death in cancer cells but not in normal cells, antitumor activity in animals with a therapeutic index of at least 2, and activation of a cell cycle checkpoint (e.g., activation or elevation of a member of the E2F family of transcription factors). As used herein, “therapeutic index” is the maximum tolerated dose divided by the efficacious dose.

In additional aspects, β-lapachone, or a pharmaceutically acceptable salt, or metabolite thereof, can be administered in combination with a second anti-cancer or anti-proliferative agent (chemotherapeutic agent). The chemotherapeutic agent can be a microtubule targeting drug, a topoisomerase poison drug or a cytidine analogue drug. In preferred aspects, the chemotherapeutic agent can be Taxol® (paclitaxel), lovastatin, minosine, tamoxifen, gemcitabine, araC, 5-fluorouracil (5-FU), methotrexate (MTX), docetaxel, vincristin, vinblastin, nocodazole, teniposide, etoposide, adriamycin, epothilone, navelbine, camptothecin, daunonibicin, dactinomycin, mitoxantrone, amsacrine, epirubicin, idarubicin, gemcitabine or imatinib.

β-lapachone, or a pharmaceutically acceptable salt, or metabolite thereof, or analog or derivative thereof (pharmaceutical composition of the invention) can be administered simultaneously with or following administration of the second anti-cancer agent or second anti-proliferative agent, more preferably the second anti-cancer agent or second anti-proliferative agent is administered following administration of the pharmaceutical composition of the invention, most preferably the second anti-cancer agent or second anti-proliferative agent is administered within 24 hours after the pharmaceutical composition of the invention is administered.

Compounds of the present invention, including β-lapachone, or a pharmaceutically acceptable salt, or metabolite thereof can be incorporated into pharmaceutical compositions suitable for administration. Such compositions typically comprise the compound (i.e. including the active compound), and a pharmaceutically acceptable excipient or carrier. As used herein, “pharmaceutically acceptable excipient” or “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field. Preferred examples of such carriers or diluents include, but are not limited to, water, saline, ringer's solutions, dextrose solution, and 5% human serum albumin. Pharmaceutically acceptable carriers include solid carriers such as lactose, terra alba, sucrose, talc, gelatin, agar, pectin, acacia, magnesium stearate, stearic acid and the like. Exemplary liquid carriers include syrup, peanut oil, olive oil, water and the like. Similarly, the carrier or diluent may include time-delay material known in the art, such as glyceryl monostearate or glyceryl distearate, alone or with a wax, ethylcellulose, hydroxypropylmethylcellulose, methylmethacrylate or the like. Other fillers, excipients, flavorants, and other additives such as are known in the art may also be included in a pharmaceutical composition according to this invention. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In one aspect, β-lapachone is administered in a suitable dosage form prepared by combining a therapeutically effective amount (e.g., an efficacious level sufficient to achieve the desired therapeutic effect through inhibition of tumor growth, killing of tumor cells, treatment or prevention of cell proliferative disorders, etc.) of β-lapachone, or a pharmaceutically acceptable salt, or metabolite thereof (as an active ingredient) with standard pharmaceutical carriers or diluents according to conventional procedures (i.e., by producing a pharmaceutical composition of the invention). These procedures may involve mixing, granulating, and compressing or dissolving the ingredients as appropriate to attain the desired preparation.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition of the invention can be administered to a subject in many of the well-known methods currently used for chemotherapeutic treatment. For example, for treatment of cancers, a compound of the invention may be injected directly into tumors, injected into the blood stream or body cavities or taken orally or applied through the skin with patches. The dose chosen should be sufficient to constitute effective treatment but not so high as to cause unacceptable side effects. The state of the disease condition (e.g., cancer, precancer, and the like) and the health of the patient should preferably be closely monitored during and for a reasonable period after treatment.

The pharmaceutical compositions containing active compounds of the present invention may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries which facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., cell cycle checkpoint activation modulator) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one aspect, the active compounds are prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In a preferred aspect, the pharmaceutically acceptable carrier can be a solubilizing carrier molecule. More preferably, the solubilizing carrier molecule can be Poloxamer, Povidone K17, Povidone K12, Tween 80, ethanol, Cremophor/ethanol, Lipiodol, polyethylene glycol (PEG) 400, propylene glycol, Trappsol, alpha-cyclodextrin or analogs thereof, beta-cyclodextrin or analogs thereof, and gamma-cyclodextrin or analogs thereof.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved.

In therapeutic applications, the dosages of the pharmaceutical compositions used in accordance with the invention vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. Dosages can range from about 0.0001 mg/kilo per day to about 1000 mg/kilo per day. In preferred aspects, dosages can range from about 1 mg/kilo per day to about 200 mg/kilo per day. An effective amount of a pharmaceutical agent is that which provides an objectively identifiable improvement as noted by the clinician or other qualified observer. Regression of a tumor in a patient is typically measured with reference to the diameter of a tumor. Decrease in the diameter of a tumor indicates regression. Regression is also indicated by failure of tumors to reoccur after treatment has stopped. As used herein, the terms “dosage effective manner” and “therapeutically effective amount” refers to amount of an active compound to produce the desired effect in a subject or cell.

In another aspect, the pharmaceutical composition can be administered at a dosage from about 2 mg/m² to 5000 mg/m² per day, preferably from about 20 mg/m² to 2000 mg/m² per day, more preferably from about 20 mg/m² to 500 mg/m² per day, most preferably from about 30 to 300 mg/m² per day. About 2 mg/m² to about 5000 mg/m² per day is the preferred administered dosage for a human.

In another aspect, the pharmaceutical composition can be administered at a dosage from about 10 to 1,000,000 μg per kilogram body weight of recipient per day; preferably about 100 to 500,000 μg per kilogram body weight of recipient per day, more preferably from about 1000 to 250,000 μg per kilogram body weight of recipient per day, most preferably from about 10,000 to 150,000 μg per kilogram body weight of recipient per day.

One of ordinary skill in the art can determine the appropriate dosage amount in mg/m² per day or μg per kilogram body weight of recipient per day depending on subject to which the pharmaceutical composition is to be administered.

The pharmaceutical compositions can be included in a container, pack, or dispenser together with instructions for administration.

The invention is further defined by reference to the following examples. It is understood that the foregoing detailed description and the following examples are illustrative only and are not to be taken as limitations upon the scope of the invention. It will be apparent to those skilled in the art that many modifications, both to the materials and methods, may be practiced without departing from the purpose and interest of the invention. Unless otherwise defined, all technical and scientific terms used herein have the same meaning as commonly understood by one of ordinary skill in the art to which this invention belongs. All publications, patent applications, patents, and other references mentioned herein are incorporated herein by reference in their entirety. In the case of conflict, the present specification, including definitions, will control.

### EXAMPLES

### Example 1

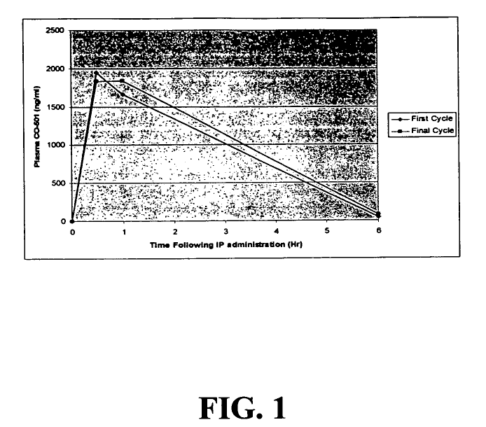

A study was performed to determine the pharmacokinetic profile of intraperitoneal (IP) dosing in mice. In the study, whole blood samples were taken following the initial β-lapachone injection (“First Cycle”) and after the eighth β-lapachone injection (“Final Cycle”). Plasma was prepared from whole blood samples and frozen pending analysis for β-lapachone concentration by LC-MS. FIG. 1 shows β-lapachone plasma concentrations following the initial IP administration of 150 mg/m² of β-lapachone formulated in Lipiodol and again after eight 3-day cycles of treatment with 150 mg/m² β-lapachone and 3 mg/m² Taxol® (both formulated in Lipiodol). In this study, peak plasma levels were achieved within one-half hour of administration, remained at approximately the same level through at least one hour post-administration, and declined to low levels at 6 hours post-dosing. No difference was evident between the initial and final dosing cycles, indicating that circulating half-life of β-lapachone does not change following repeated dosing. The peak plasma level corresponds to approximately 8 μM of β-lapachone. Animals showed no evident toxicity in this study.

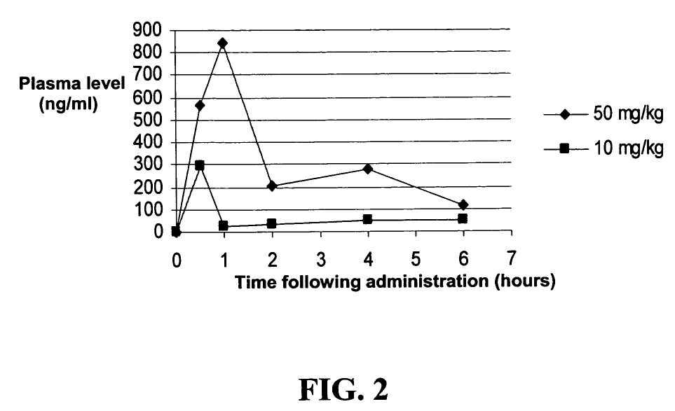

Similar studies have been conducted in non-tumor bearing female nude (Ncr) mice using β-lapachone formulated in hydroxypropyl-α-cyclodextrin (HPBCD). The preferred formulation is 10 mg/mL 3-lapachone in 40% HPBCD. Nude mice received 50 mg/kg or 10 mg/kg intraperitoneally. At each time point, whole blood samples were taken via cardiac puncture from 3 female mice (NCR) following a single β-lapachone IP injection. Plasma was prepared from whole blood samples and frozen pending analysis for β-lapachone concentration by LC-MS. The results displayed in FIG. 2 and Table 1 show that peak serum levels are lower with the aqueous HPBCD formulation than with the Lipiodol formulation, but that the area under the curve (AUC) is approximately the same over 6 hours. Table 1 compares the AUC and Cₘₐₓ achieved with Lipiodol and HPBCD formulations.

TABLE 1

| Formulation | AUC* | Cₘₐₓ** |
|---|---|---|
| Lipiodol formulation | 2382 | 2744 |
| (50 mg/kg) | | |
| HPBCD formulation | 1903 | 844 |
| (50 mg/kg) | | |
| HPBCD formulation | 358 | 294 |
| (10 mg/kg) | | |

*ng-hr/ml
**ng/ml

Since in vivo efficacy with both formulations is equivalent despite significant differences in Cₘₐₓ, the data shows that efficacy correlates with AUC. These studies indicate that the AUC range can be from about 0.5 to about 100 (μM-hr), the Cₘₐₓ range from about 0.1 to about 100 (μM) and the dosage from about 2 to about 5000 (mg/m²). This is consistent with in vitro data which indicates that total drug exposure is more closely related to induction of apoptosis than peak drug concentration. Thus efficacy in animals can be achieved by doses of β-lapachone in the clinical formulation that result in total exposure to the drug of approximately 0.5 to about 100 μM-hr, corresponding to administered doses of 2 to about 5000 mg/m².

Prior studies have shown that 1 to 2 μM applied to cells for approximately four hours (exposure of 4 to 8 μM-hr/ml) reaches or exceeds the in vitro LC₅₀ for many cancer cell lines. Therefore, the pharmacokinetic data is consistent with in vitro cytotoxicity results, confirming that doses of β-lapachone in the clinical formulation that result in total exposure to the drug of approximately 0.5 to about 100 μM-hr, corresponding to administered doses of 2 to about 5000 mg/m² are essential for therapeutic treatment in patients.

### Example 2

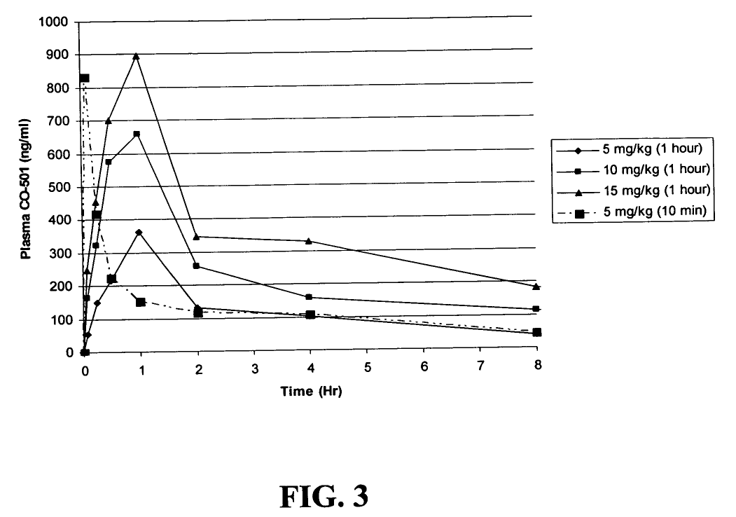

The pharmacokinetics of β-lapachone following intravenous administration in both rats and dogs have been evaluated. The results following intravenous infusion of low doses of β-lapachone for one hour or ten minutes in the HPBCD formulation to male Sprague-Dawley rats are shown in FIG. 3 and Table 2. Plasma concentrations of β-lapachone were determined by LC-MS. There were no signs of toxicity at these doses. These studies indicate that the AUC is linear and the elimination half-life is 4.5-7 hours in the rat following a one-hour infusion of doses in the range 5-15 mg/kg. As would be expected from a hydrophobic drug substance, the volume of distribution is large (20-26 L/kg).

TABLE 2

| Parameter | 1-Hour Infusion | | | 10-Min. Infusion |
|---|---|---|---|---|
| Dose | 5 mg/kg | 10 mg/kg | 15 mg/kg | 5 mg/kg |
| AUC (ng-hr/ml) | 1285 ± 118 | 2645 ± 1022 | 4898 ± 54 | 1198 ± 313 |
| Cmax (ng/ml) | 363 ± 21 | 707 ± 134 | 896 ± 130 | 834 ± 116 |
| T_{1/2} (Hr) | 4.3 ± 0.6 | 4.9 ± 1.6 | 7.0 ± 1.6 | 3.6 ± 0.9 |
| Vdss (L/kg) | 19.6 ± 0.8 | 21.0 ± 5.3 | 26.4 ± 5.7 | 20.0 ± 6.3 |

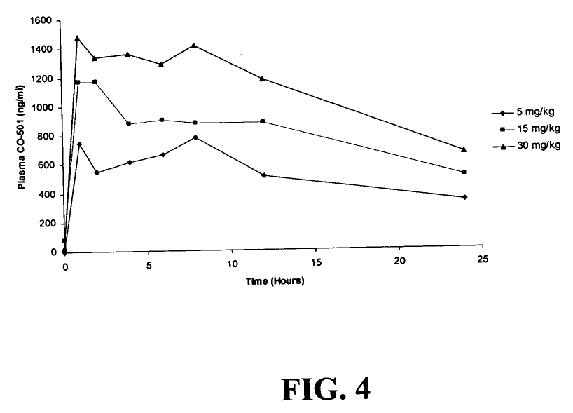

Pharmacokinetic data from a toxicology study in dogs are shown in FIG. 4 and Table 3. In this study, two dogs (one male, one female) were administered β-lapachone intravenously in the HPBCD formulation over one hour. The animals received escalating doses of 5, 15, 30 and 45 mg/kg, with three to four days between doses. The mean response of the two dogs is shown at each dose level. Plasma concentrations of β-lapachone were determined by LC-MS. Doses up to and including 30 mg/kg produced no toxicity. Animals were euthanized before completion of the 45 mg/kg dose, which produced severe toxic signs.

In the dogs, the distribution phase was slow, with plasma levels remaining relatively preserved for the first 8 hours after dosing. The terminal half-life was approximately 16 hours across the dose range. These studies demonstrate that intravenous administration of β-lapachone to rats and dogs readily achieves therapeutic levels without evidence of toxicity.

TABLE 3

| | Dose | | | | | |
|---|---|---|---|---|---|---|
| Parameter | 5 mg/kg | | 15 mg/kg | | 30 mg/kg | |
| Infusion Time | 60 | min | 60 | min | 60 | min |
| AUC (ng-hr/ml) | 13,575 | | 20,225 | | 26,000 | |
| Cmax (ng/ml) | 750 | | 1,150 | | 1,475 | |
| T_{1/2} (Hr) | ˜16 | hr | ˜16 | hr | ˜16 | hr |

### Example 3

The extent of binding of β-lapachone to human plasma proteins was measured by means of equilibrium dialysis against PBS buffer at 37° C. β-lapachone formulated in 40% HPBCD was added to pooled human plasma aliquots to final concentrations of 2, 5, 10, 17 and 25 μM; each plasma aliquot contained 70,000˜90,000 DPM of ¹⁴C-labeled β-lapachone. The plasma aliquots were incubated at 37° C. for 1 h, and then were dialyzed against PBS buffer at 37° C. for 4 h using Dianorm Equilibrium Dialysers. The distribution of β-lapachone between the plasma and the PBS was then determined by quantitating the ¹⁴C-labeled β-lapachone in both solutions, and then the free β-lapachone fraction in human plasma was calculated using the method of Hu and Curry (Biopharm Drug Dispos. 1986 March-April; 7(2):211-4). Since previous studies showed that β-lapachone is stable to degradation when incubated in human plasma at 37° C. for 16 hrs, no adjustments were made for β-lapachone degradation in plasma. The results of the study showed that the amount of free 1-lapachone in human plasma is approximately 7% (93% protein binding) over the entire range of concentrations studied (2-25 μM).

The reversibility of the binding of β-lapachone to human plasma proteins was assessed in a similar experiment. β-lapachone formulated in 40% HPBCD was added to pooled human plasma to a final concentration of 10 μM; the plasma aliquot contained 70,000˜90,000 DPM of ¹⁴C-labeled β-lapachone. The spiked plasma was incubated at 37° C. for 1 h, and then was dialyzed three times against fresh plasma aliquots at 37° C. The distribution of 1-lapachone between the spiked plasma and the fresh plasma was determined by quantitating the ¹⁴C-labeled β-lapachone.

This study showed that at least 65% of 1-lapachone in plasma was reversibly bound to plasma proteins and could be dialyzed into fresh human plasma. Since the dialysis was not exhaustive, the actual amount of reversibly bound 1-lapachone is higher, likely approaching 100%.

### Example 4

Numerous toxicology studies have been conducted with β-lapachone and are described herein. The GLP and toxicology studies performed for β-lapachone are shown below in Tables 4 and 5, respectively. Calvert Preclinical Services, Inc. Olyphant, Pa. performed all animal studies except for the cardiovascular study in dogs, which was performed by MDS Pharma Services-Taiwan Ltd., Taipei, Taiwan.

TABLE 4

| Study No. | Title |
|---|---|
| 1A | A Repetitive Weekly Intravenous Toxicity Study of |
| | β-Lapachone in Rats |
| 2A | A Repetitive Weekly Intravenous Toxicity Study of |
| | β-Lapachone in Dogs |
| 3A | Neuropharmacological Profile of β-Lapachone in Rats |

TABLE 5

| Study No. | Title |
|---|---|
| 1B | A Dose-Range-Finding Single Dose Intravenous Toxicity |
| | Study of a Test Article in Rats |
| 2B | A Dose-Range-Finding Toxicity Study of β-Lapachone |
| | Following Single Intravenous Administration in Dogs |
| 3B | Acute Toxicity Study of β-Lapachone Following Single |
| | Intravenous Infusion in Rats |
| 4B | Cardiovascular, Pulmonary Study of β-Lapachone in |
| | Anesthetized Dogs |
| 5B | A Four Week Intravenous Toxicity Study of β-Lapachone |
| | in Rats |
| 6B | A Two Week Intravenous Repetitive Dose Toxicity Study of |
| | β-Lapachone in Rats |

β-lapachone drug product was provided to the contract test laboratories in the HPBCD formulation for intravenous infusion. The formulation is comprised of 10 mg/ml β-lapachone in 40% HPBCD. The drug product was diluted to dosing concentrations with 0.45% or 0.9% NaCl prior to infusion.

Design features of the individual studies are summarized in Tables 6 and 7.

TABLE 6
Overview of GLP toxicology studies

| Title | | | M/F per | | | |
|---|---|---|---|---|---|---|
| (Study ID #) | Species | No. of Groups | Group | Doses | Regimen | Parameters |
| A Repetitive Weekly | Rat | Toxicology: 4 | 15 M/15 F in | 0, 10, 25, | Dose q7d × 4, 1 | Mortality |
| Intravenous Toxicity | | Toxicokinetic: 4 | control and | 45 mg/kg | hour intravenous | Clinical Signs |
| Study of β-Lapachone | | | high-dose | | infusion via tail vein | Body Weights |
| in Rats | | | groups; | | Sac day 23 - 10 | Food |
| (1A) | | | 10 M/10 F in | | animals/sex/group | Consumption |
| | | | low-and | | Sac day 37 - 5 | Hematology |
| | | | mid-dose | | animals/sex in control | Coagulation |
| | | | groups | | and high dose groups | Clin Chem |
| | | | 9 M/9 F | | | Gross Pathology |
| | | | | | | Histopathology |
| | | | | | | Toxicokinetics |
| A Repetitive Weekly | Dog | 4 | 6 M/6 F in | 0, 5, 15, 35 | Dose q7d × 4, 1 | Mortality |
| Intravenous Toxicity | | | control and | mg/kg | hour intravenous | Clinical Signs |
| Study of β-Lapachone | | | high-dose | | infusion via cephalic | Body Weights |
| in Dogs | | | groups; | | vein | Food |
| (2A) | | | 4 M/4 F in | | Sac day 23 - 4 | Consumption |
| | | | low- and | | animals/sex/group | Hematology |
| | | | mid-dose | | Sac day 37 - 2 | Coagulation |
| | | | groups | | animals/sex in control | Clin Chem |
| | | | | | and high dose groups | Gross Pathology |
| | | | | | | Histopathology |
| | | | | | | Toxicokinetics |
| Neuropharmacological | Rat | 4 | 10 M | 0, 5, 25, 60 | Dose × 1, 1 hour | CNS Signs |
| Profile of β-Lapachone | | | | mg/kg | intravenous infusion | Body |
| in Rats | | | | | via tail vein | Temperatures |
| (3A) | | | | | Sac 24 hr post | |
| | | | | | dosing | |

TABLE 7
Overview of toxicology studies

| Title | | No. of | M/F per | | | |
|---|---|---|---|---|---|---|
| (Study ID #) | Species | Groups | Group | Doses | Regimen | Parameters |
| A Dose-Range-Finding Single | Rat | 5 | 3 M/3 F | 0, 30, 45, | Dose × 1, 1 hour | Mortality |
| Dose Intravenous Toxicity Study | | | | 60, 75 | intravenous infusion | Clinical Signs |
| of a Test Article in Rats | | | | mg/kg | via tail vein | Body Weights |
| (1B) | | | | | Sac day 8 | Food |
| | | | | | | Consumption |
| | | | | | | Hematology |
| | | | | | | Coagulation |
| | | | | | | Clin Chem |
| | | | | | | Gross Pathology |
| | | | | | | Histopathology |
| A Dose-Range-Finding Toxicity | Dog | 1 | 1 M/1 F | 5, 15, 30, | Escalating dose | Mortality |
| Study of β-Lapachone Following | | | | 45 | given every 3-4 days, | Clinical Signs |
| Single Intravenous | | | | mg/kg/day | intravenous infusion | Body Weights |
| Administration in Dogs | | | | | via cephalic vein | Food |
| (2B) | | | | | Sac within 48 h of | Consumption |
| | | | | | final dose | Hematology |
| | | | | | administration | Clin Chem |
| | | | | | | Gross Pathology |
| | | | | | | Histopathology |
| | | | | | | Toxicokinetics |
| Acute Toxicity Study of β- | Rat | 1 | 3 M | 75 mg/kg | Dose × 1, 1 hour | Mortality |
| Lapachone Following Single | | | | | intravenous infusion | Clinical Signs |
| Intravenous Infusion in Rats | | | | | via tail vein | Hematology |
| (3B) | | | | | Sac day 1 | Coagulation |
| | | | | | | Clin Chem |
| Cardiovascular, Pulmonary | Dog | 3 | 3 M or F | 0, 20, 40 | Dose × 1, 1 hour | ECG (QTc, PRI, |
| Study of β-Lapachone in | | | | mg/kg | intravenous infusion | QRS, ST) |
| Anesthetized Dogs | | | | | via cephalic vein | HR |
| (4B) | | | | | Sac day 1 | BP |
| | | | | | | BF |
| | | | | | | TV |
| | | | | | | RR |
| | | | | | | RL |
| | | | | | | CDYN |
| A Four Week Intravenous | Rat | 3 | 3 M | 0, 30, 45 | Dose q7d × 4, 1 | Mortality |
| Toxicity Study of β-Lapachone | | | | mg/kg | hour intravenous | Clinical Signs |
| in Rats | | | | | infusion via tail vein | Body Weights |
| (5B) | | | | | Sac day 24 | Food |
| | | | | | | Consumption |
| | | | | | | Hematology |
| | | | | | | Clin Chem |
| | | | | | | Gross Pathology |
| | | | | | | Histopathology |
| A Two Week Intravenous | Rat | 2 | 3 M | 0, 45 mg/kg | Dose qd × 5, 1 hour | Mortality |
| Toxicity Study of β-Lapachone | | | | | intravenous infusion | Clinical Signs |
| in Rats | | | | | via tail vein | Body Weights |
| (6B) | | | | | If no clinical signs | Food |
| | | | | | of toxicity, begin | Consumption |
| | | | | | dose bid; if clinical | Hematology |
| | | | | | signs, begin dose q2d | Coagulation |
| | | | | | Sac day 15 | Clin Chem |
| | | | | | | Gross Pathology |
| | | | | | | Histopathology |

The results of the study indicate that therapeutic plasma concentrations of β-lapachone are attainable in humans following administration of doses in the range of 10-40 mg/m². Our studies have focused on exploring the toxicity of multiples of these doses. Further, the results of the studies, described in more detail in Examples 5-14, show:
Acute single dose escalation studies have shown that one hour IV infusions are generally well tolerated up to and including 270 mg/m² (45 mg/kg) in the rat and 535 mg/m² (30 mg/kg) in the dog.
Repetitive weekly one-hour intravenous infusions x 4, modeled after the intended clinical route and dosing interval, is without significant toxicity at doses less than 270 mg/m² in rats and dogs. At or above this level, principal findings were a small decrease in hemoglobin with reticulocytosis and mildly elevated bilirubin levels which both revert to normal during 2-week recovery.
Lethality has been observed in rats following one hour intravenous infusions of 360 mg/m² (60 mg/kg) and in dogs at 800 mg/m² (45 mg/kg). The cause of death is not evident from gross or microscopic pathology. Lethality may be due to non-specific effects of infusion of highly concentrated drug solution on injection site tissue and blood.
No evidence of major organ toxicity including myelosuppression, hepatic, renal, neurotoxicity or cardiotoxicity occurs at any dose or regimen tested.

Consistent with the high degree of selectivity expected of β-lapachone based on its mechanism of action, no significant irreversible toxicity has been identified at multiples of the anticipated therapeutic dose. Above these doses, the first manifestation of toxicity in both rats and dogs is a reversible drop in hemoglobin with reticulocytosis, hyperbilirubinemia and hemosiderosis, suggestive of extravascular hemolysis. These findings resolved during the two week recovery period in both species.

Above these doses, at toxic levels, muscular weakness with resultant respiratory insufficiency occur, which are reversible if short term lethality does not occur. The cause of this toxicity is thought to be local effects of high drug concentrations on injection site tissues (including blood) which results in systemic effects not related to the pharmacology of the drug; however, other etiologies have not been ruled out.

β-lapachone shows no evidence of bone marrow suppression, gastrointestinal toxicity or alopecia. There was also no evidence of any specific major organ toxicity related to heart, circulatory system, nervous system, liver, or kidneys.

The no-observed effect level (NOEL) for β-lapachone administered in four weekly one-hour intravenous infusions is 60 mg/m² (10 mg/kg) in rats and 90 mg/m² (5 mg/kg) in dogs. The STD is >270 mg/m² (45 mg/kg) in rats and >625 mg/m² (35 mg/kg) in dogs.

### Example 5

GLP toxicology studies in rat and dog encompass a broad range of doses of β-lapachone administered by one hour infusion at weekly intervals which mimics administration of β-lapachone in humans. Dose ranges were designed to elicit sub-lethal toxicity at the high doses based on results of toxicology studies. Doses used are shown in Table 8. Note that the low doses used in these studies produce supra-therapeutic plasma levels of β-lapachone.

TABLE 8

| | Rat | | | | Dog | | | |
|---|---|---|---|---|---|---|---|---|
| | Study 1A | | | | Study 1B | | | |
| Dose | 0 | 10 | 25 | 45 | 0 | 5 | 15 | 35 |
| (mg/kg) | | mg/kg | mg/kg | mg/kg | | mg/kg | mg/kg | mg/kg |
| Dose | 0 | 60 | 150 | 270 | 0 | 90 | 270 | 625 |
| (mg/m²) | | mg/m² | mg/m² | mg/m² | | mg/m² | mg/m² | mg/m² |
| AUC* | | 2,645 | ND | ND | | 13,575 | ND | ND |
| | | ng-hr/mL^{#} | | | | ng-hr/mL^{#} | | |

*Estimated from toxicology experiments. Analysis of concurrent toxicokinetic samples currently under way.
^{#}Estimated therapeutic AUC = approximately 1500 ng-hr/ml (6.5 μM-hr/mL)

### Example 6

In Study No. 1A, adult male and female Sprague-Dawley Hsd:SD rats were randomized into treatment groups and received doses of 0 (vehicle), 10, 25 and 45 mg/kg β-lapachone via intravenous infusion as shown below in Tables 9 and 10.

TABLE 9
Toxicology Groups

| | | Dosage | Concen- | Dose | Number of |
|---|---|---|---|---|---|
| Group | | Level | tration | Volume | Males/ |
| No. | Treatment | (mg/kg/day) | (mg/mL) | (mL/kg/day) | Females |
| 1 | Control | 0 | 0 | 15.0 | 15/15 |
| 2 | Low-dose | 10 | 0.67 | 15.0 | 10/10 |
| 3 | Mid-dose | 25 | 1.67 | 15.0 | 10/10 |
| 4 | High-dose | 45 | 3.0 | 15.0 | 15/15 |

TABLE 10
Toxicokinetic Groups

| | | Dosage | Concen- | Dose | Number of |
|---|---|---|---|---|---|
| Group | | Level | tration | Volume | Males/ |
| No. | Treatment | (mg/kg/day) | (mg/mL) | (mL/kg/day) | Females |
| 5 | Low-dose | 10 | 0.67 | 15.0 | 9/9 |
| 6 | Mid-dose | 25 | 1.67 | 15.0 | 9/9 |
| 7 | High-dose | 45 | 3.0 | 15.0 | 9/9 |

β-lapachone, provided as the HPBCD formulation (10 mg/ml), was diluted to the dosing concentration with 0.9% NaCl. The β-lapachone concentration in the dosing solutions was confirmed by spectrophotometric analysis. The β-lapachone dosing solutions were administered weekly (Days 1, 8, 15 and 22) to each rat in each toxicology and toxicokinetic group as an intravenous infusion over 1 hour into the tail vein. Each animal was dosed based upon its most recent body weight.

Blood samples for hematology and serum chemistry (maximum 2.5 mL per animal) were taken from all toxicology study animals (Groups 14) via the lateral tail vein on Day 2. Blood samples for hematology, coagulation and serum chemistry were collected via cardiocentesis on Day 23 (10 animals/sex/group) or Day 37 (all surviving animals) prior to sacrifice. Whole blood was collected from 10 animals/sex from animals not assigned to the study for baseline values.

Blood samples (0.5 mL) for toxicokinetic evaluation were taken from toxicokinetic group animals (Groups 5-7) by retroorbital puncture on Days 1 and 22 at the following timepoints: pre-treatment, 1, 2, 4, 8, 12 and 24 hours post-infusion start. Blood samples were taken from 3 animals/sex/group at each time point, with no animal being bled more than 3 times in any 24 hour period. Toxicokinetic animals were euthanized following their final blood collection.

On Day 23 of the study, 10 animals/sex/group were euthanized (CO₂ asphyxiation) and subjected to necropsy as described below. The remaining animals in Groups 1 and 4 remained on study, untreated, until sacrifice on Day 37.

The principle clinical observation made during the study was the appearance of swollen, irritated tails principally among high dose animals following administration of the first dose of study drug. Following administration of subsequent doses, administration needles were flushed with a small amount of saline prior to removal. This significantly reduced incidence of the finding on subsequent doses, however considerable tissue inflammation persisted in the tails of a number of high-dose animals.

There was no mortality in any group among the toxicokinetic animals. Among the toxicology animals, no mortality or clinical signs of toxicity occurred in the control, low- or mid-dose groups. One animal in the 45 mg/kg toxicology high dose group was found dead on Day 3 with histopathologic findings of mild multifocal myocardial necrosis with right atrial dilatation and congestion, lesions not seen in other animals in the study. Relationship to study drug could not be established. Two other high dose animals were sacrificed on Day 14 and Day 15. One animal was sacrificed when in the judgment of the study veterinarian the inflamed condition of the animal's tail put it at significant risk of systemic infection. Following sacrifice this animal had histopathologic changes similar to other high dose animals in the study (see below). The other animal was sacrificed due to loss of body weight. This animal had non-specific histopathological findings related to stress, not considered to be directly test article related.

Clinical pathology was generally unremarkable in all groups except for small (<2 g/dl) decreases in hemoglobin seen in mid- and high-dose animals; these findings normalized by Day 37. Slight increases in bilirubin (eg 0.33 mg/dl, ULN 0.27 mg/dl) were seen in high dose animals at Days 2 and 23 but were also normal on Day 37. Slight increases in CK and AST were seen in high dose animals after the first dose on Day 2 but were normal on Day 23 following completion of all doses and on Day 37.

Histopathological evaluation of high dose animals showed hemosiderosis and extramedullary hematopoiesis in liver and spleen. These changes were seen at a milder degree in mid-dose animals. The injection site in high dose animals showed moderate to marked inflammatory changes with inflammation and necrosis of surrounding tissues. A subtle axonopathy of the sciatic nerve may have resulted from local extension of tail inflammatory and/or vascular changes. Histopathologic changes resolved by Day 37 other than tail vein findings, which partially resolved.

The results of this study show that β-lapachone is well tolerated in male Sprague Dawley rats when administered as four weekly one-hour intravenous infusions at doses up to and including 45 mg/kg. At this top dose, a small drop in hemoglobin, slightly elevated bilirubin and histopathologic findings were seen, compatible with mild extravascular hemolysis of uncertain pathogenesis which resolved when drug was stopped. Based on the results of this study, the NOEL was 10 mg/kg/dose.

### Example 7

In Study No. 2A, adult male and female Beagle dogs were randomized to treatment groups and dosed with 4×1 hour intravenous infusions at doses of 0 (vehicle), 5, 15 and 35 mg/kg as shown in Table 11.

TABLE 11

| | | Dosage | Concen- | Dose | Number of |
|---|---|---|---|---|---|
| Group | | Level | tration | Volume | Males/ |
| No. | Treatment | (mg/kg/day) | (mg/mL) | (mL/kg/day) | Females |
| 1 | Control | 0 | 0 | 10.0 | 6/6 |
| 2 | Low-dose | 5 | 0.5 | 10.0 | 4/4 |
| 3 | Mid-dose | 15 | 1.5 | 10.0 | 4/4 |
| 4 | High-dose | 35 | 3.5 | 10.0 | 6/6 |

β-lapachone, provided as the HPBCD formulation (10 mg/ml), was diluted to the dosing concentration with 0.9% NaCl. The β-lapachone concentration in the dosing solutions was confirmed by spectrophotometric analysis. The β3-lapachone dosing solutions were administered weekly (Days 1, 8, 15 and 22) to each dog as an intravenous infusion over 1 hour into the cephalic vein. Each animal was dosed based upon its most recent body weight.

Blood samples for hematology, coagulation and serum chemistry (maximum 20 mL per animal per day) were collected via the jugular vein prior to treatment initiation, on Day 2 and on Day 23 or Day 37. Animals were fasted 12-24 hours prior to blood collection, except on Day 2 when animals were not fasted prior to blood collection. Blood samples (approximately 1 mL/sample) for toxicokinetic evaluation were collected from the jugular vein of each animal on Days 1 and 22 at the following time points: immediate pre-treatment, 1, 2, 4, 6, 8, 12 and 24 hours post-infusion start.

On Day 23 of the study, 4 animals/sex/group were euthanized (barbiturate overdose) followed by exsanguination and subjected to necropsy as described below. The remaining animals in Groups 1 and 4 remained on study, untreated, until sacrifice on Day 37.

There were no early deaths during the study. Minimal clinical signs were noted in the control and low dose groups. In the mid-dose group, transient clinical signs were occasionally noted post-dosing but resolved within 24 hours. These included decreased activity, abnormal gait and stance, salivation and swelling at the injection site. In the high-dose group, similar findings occurred along with signs of discomfort during drug administration, including, vocalization, head thrashing, tremors, body thrashing and occasional emesis. Typically, clinical signs in high-dose animals resolved within 24 hrs.

Body weight and food consumption in low- and mid-dose groups were comparable to controls. High-dose animals showed a slight mean decrease in body weight (−7% in males and −4% in females) that generally correlated with reduced food consumption; during the recovery period, body weights recovered to levels comparable to the controls.

There were no drug-related ophthalmologic findings.

The clinical pathology data show decreased hemoglobin in mid-dose (˜3 g/dl) and high-dose (˜5 g/dl) animals with reticulocytosis at Day 23. These values returned to normal during the recovery period. A dose-dependent slight increase in bilirubin was seen at Days 2 and 23, and also resolved by Day 37.

The only significant gross pathologic observation was splenic enlargement in mid- and high-dose animals (5/8 and 3/8, respectively), which correlated with microscopic findings of dose-related extramedullary hematopoiesis and hemosiderosis in liver and spleen. These partially reversed during the 14-day recovery. Thymus in high dose animals showed involution consistent with nonspecific stress reaction. In the high dose group adrenal glands showed cortical atrophy and other changes compatible with stress reaction.

Preliminary toxicokinetic results show that the plasma levels of β-lapachone in mid- and high-dose animals were significantly above the values predicted from a dose-range finding pilot study. Detailed pharmacokinetic analysis of these data is pending and will be submitted when available.

The results of these studies show that administration of four weekly intravenous doses of β-lapachone is well-tolerated in dogs at 5 mg/kg. At 15 mg/kg and 35 mg/kg, signs of reversible extravascular hemolysis were evident, along with a variety of transient clinical signs. The drop in hemoglobin was somewhat greater than seen in the rat study, but doses were higher (mg/m²), and animals underwent greater phlebotomy since separate toxicokinetic groups were not utilized. Animals receiving 35 mg/kg showed stress reactions in adrenal and thymus.

### Example 8

In Study No. 3A, four groups of ten male Sprague-Dawley rats were dosed with a single 1 hour intravenous infusion via the tail vein at doses of 0 (vehicle), 5, 25, and 60 mg/kg as shown in Table 12.

TABLE 12

| | | Dosage | Concen- | Dose | Number of |
|---|---|---|---|---|---|
| Group | | Level | tration | Volume | Animals |
| No. | Treatment | (mg/kg) | (mg/ml) | (ml/kg) | (Male) |
| 1 | Vehicle | 0 | 0 | 10.0 | 10 |
| 2 | β-Lapachone | 5 | 0.5 | 10.0 | 10 |
| 3 | β-Lapachone | 25 | 2.5 | 10.0 | 10 |
| 4 | β-Lapachone | 60 | 6 | 10.0 | 10 |

Following dosing, animals were placed in a fixed environment consisting of a Plexiglas square, fitted with a lid and placed on absorbent paper to detect excretions. The rats were observed for signs of pharmacological or toxicological activity at 5, 15, and 30 minutes, 1, 2, 3, 4, and 24 hours following treatments. Observances were made for the symptoms listed in Table 13.

TABLE 13

| | |
|---|---|
| Seizures/convulsions | Awareness reaction |
| Startle response | Vocalization |
| Irritability | Decreased abdominal tone |
| Increased secretion | Body tremors |
| Decreased grip strength | Immobility |
| Motor activity | Ataxia |
| Abnormal posture | Sterotypy |
| Excretion | Decreased respiration |
| Piloerection | Loss of righting |
| Pupil size | Nociceptive (pain) response |
| Corneal reflex | Pinnal reflex |

Body temperatures were taken on all animals at 15 minutes following dose administration.

Animals dosed at 0, 5 and 25 mg/kg β-lapachone showed no pharmacological or toxicological signs throughout the 24 hours post-dose. Five of the ten animals receiving the 60 mg/kg dose, which was dosed at a concentration of 6 mg/ml, died during the infusion. Labored respiration was noted for all ten animals during the infusion. The surviving animals typically showed labored respiration, abnormal gait, decreased activity, and decreased abdominal tone for 1-4 hours post infusion. All surviving animals recovered by 24 hours post dosing. Also in this dose group mean body temperature 15-minutes post dosing was 34.6° C., approximately 3° C. lower than the other three treatment groups.

The results of this study show that the NOEL for β-lapachone is 25 mg/kg when administered as a single intravenous dose to male Sprague Dawley rats. Signs in animals receiving 60 mg/kg suggested muscular weakness with respiratory compromise. The origin of the hypothermia is not clear from this study, although it may be secondary to reduced muscular activity. Other neurological signs were not observed. A dose of 60 mg/kg, which was well tolerated in Sprague Dawley rats in a previous study, caused lethality in this study, suggesting that 60 mg/kg is close to a threshold dose in the rat. Clinical signs in surviving animals resolved within 24 hours, indicating that the acute toxicity is largely reversible if the animal survives the exposure.

### Example 9

In Study No. 1B, five groups of six adult Sprague-Dawley rats (three male and three female) were randomized to treatment groups and treated via intravenous infusion as shown in

TABLE 14

| | | | Dose | Number of | |
|---|---|---|---|---|---|
| Group | Dosage Level | Concentration | Volume | Animals | |
| Treatment | (mg/kg) | (mg/ml) | (ml/kg) | Male | Female |
| Vehicle | 0 | 0 | 15.0 | 3 | 3 |
| Low-Dose | 30 | 2.0 | 15.0 | 3 | 3 |
| Mid-Dose-1 | 45 | 3.0 | 15.0 | 3 | 3 |
| Mid-Dose-2 | 60 | 4.0 | 15.0 | 3 | 3 |
| High-Dose | 75 | 5.0 | 15.0 | 3 | 3 |

The β-lapachone dosing solutions were administered to each rat, based on most recent body weight, as a single intravenous infusion over 1 hour into the tail vein.

Animals were observed for seven days post dosing. On Day 2, blood samples were taken via retro-orbital puncture (at approximately 24 hours post-dose) for hematology and clinical chemistry evaluation. Other observations included clinical signs, body weights and food consumption. On Day 8, animals were anesthetized with CO₂ and blood for testing was collected by cardiocentesis, then animals were euthanized by CO₂ asphyxiation and subjected to gross necropsy with histopathological evaluation performed on major organs (lungs, heart, liver, brain, spleen and kidneys).

No mortality or clinical signs of toxicity were noted in the control, 30 or 45 mg/kg dose groups. There was no mortality in the 60 mg/kg dose group, but transient clinical signs developed (rapid respiration, abnormal gait and stance) 1-2 hours after completion of dosing and resolved by Day 2.

In the 75 mg/kg dose group, similar clinical signs were noted immediately after dose administration and five of the six animals expired within one hour of completion of dosing. One male animal survived but continued to display signs on Days 24. The condition of the animal improved somewhat during the one week post-dose observation period.

Hematology and serum chemistry values were generally unremarkable in all groups.

No macroscopic abnormalities of internal organs were noted on gross necropsy. Histopathological evaluation showed no significant findings in the animals dosed at 0, 30, 45 or 60 mg/kg. Minimal to mild hepatocellular vacuolation and renal tubular epithelial pallor and attenuation were the only histopathological findings in the 75 mg/kg group. These findings were not evident in the animal that survived 75 mg/kg dosing and was sacrificed on Day 8.

The results in this study show that the NOEL for β3-lapachone was 45 mg/kg when administered as a single one-hour intravenous dose at a concentration of 3 mg/ml to male and female Sprague Dawley rats. The higher dose of 60 mg/kg, administered at a drug concentration of 4 mg/ml, was tolerated with no lasting clinical signs and no laboratory evidence of toxicity. The hepatocellular vacuolation and renal tubular pallor likely reflect terminal events in the high dose group.

### Example 10

In Study No. 3B, one group of three male Sprague-Dawley rats was dosed with a single 1 hour intravenous infusion via tail vein at a dose of 75 mg/kg in a dose volume of 10 ml/kg. Another group of three untreated control male animals was used for comparison of laboratory values. Details of the treatment group are shown Table 15.

TABLE 15

| | Dosage | | | |
|---|---|---|---|---|
| Group | Level | Concentration | Dose Volume | Number of |
| Treatment | (mg/kg) | (mg/ml) | (ml/kg) | Males |
| β-Lapachone | 75 | 7.5 | 10 | 3 |

Animals were observed for mortality and clinical signs during dosing. Immediately after dose completion, animals were exposed to CO₂ and blood samples for hematology and serum chemistry were taken by cardiocentesis. Animals were then euthanized (CO₂ asphyxiation) and discarded. Prior to dosing, blood was also taken from three naïve colony animals for comparison.

One of the three rats died during the β-Lapachone infusion (2.4 ml of the total 2.9 ml dose was administered). All animals showed labored respiration during dosing. The two surviving animals had decreased body tone and abnormal gait following dosing. No significant changes were seen in hematological parameters. Bilirubin was mildly elevated (0.5 mg/dl vs 0.12 mg/dl control). There were no significant changes in serum phosphorus, calcium, magnesium, hematology, or other serum chemistries.

In this study, 75 mg/kg, administered at a drug concentration of 7.5 mg/ml, was lethal for 1 of 3 animals. Clinical signs were consistent with muscular weakness. A decrease in phosphorus levels was not seen in rats. The significance of elevated bilirubin in the absence of changes in liver enzymes or hematological parameters is not evident.

### Example 11

In Study No. 2B, two adult Beagle dogs (one male and one female) were treated as shown in Table 16.

TABLE 16

| Group | Dosage Level | Concentration | Dose Volume |
|---|---|---|---|
| Treatment | (mg/kg/day) | (mg/ml) | (ml/kg) |
| β-Lapachone | 5 | 0.5 | 10.0 |
| β-Lapachone | 15 | 1.5 | 10.0 |
| β-Lapachone | 30 | 3.0 | 10.0 |
| β-Lapachone | 45 | 4.5 | 10.0 |

β-lapachone dosing solutions were administered to each dog, based on most recent body weight, as a single intravenous infusion over 1 hour into the cephalic vein. Dose levels were increased until evidence of severe toxicity was noted. The time interval between each dose was three to four days.

Animals were observed for clinical signs, and body weights and food consumption were recorded. Blood samples were collected for hematology and clinical chemistry from fasted animals via jugular vein prior to dose administration, at approximately one hour post-dose, approximately 24 hours post-dose and prior to terminal sacrifice. Whole blood samples (1 ml/sample) were also collected on each day of dose administration for toxicokinetics analysis.

There were no test article-related effects on body weight or food consumption during the study. No clinical signs of toxicity were noted at doses of 5, 15 or 30 mg/kg. Clinical signs occurred during administration of the 45 mg/kg dose, including labored breathing, vocalization, and other signs of distress with lack of pain response and prostration. Dosing was halted shortly prior to the completion of dosing and animals were euthanized. Gross necropsy revealed pale and/or discolored mucous membranes and internal organs.

No significant changes were noted in serum chemistry parameters at doses of 5, 15 and 30 mg/kg. At the 45 mg/kg dose, total bilirubin levels increased from predose values of 0.38 and 0.26 mg/dl to 0.66 and 0.87 mg/dl. Phosphorus levels were reduced from pre-dose values of 5.8 and 5.2 mg/dl to 2.6 and 0.8 mg/dl with no change in (total) serum calcium. Other serum chemistries showed no significant abnormalities.

Over the course of the 11-day study, the hemoglobin of both animals gradually declined from 14.9 and 13.5 to 9.1 and 9.7 g/dl. Anisocytosis and mild polychromasia were noted prior to administration of the 45 mg/kg dose (3 days after the 30 mg/kg dose).

The results of this study showed that intravenous infusions of β-lapachone up to and including 30 mg/kg over 1 hour have no significant acute toxicologic effects in beagle dogs. Toxicity occurred at the 45 mg/kg dose. Clinical effects (e.g. respiratory effects, prostration) and results from gross necropsy (pale tissues and discolored organs) were compatible with muscular weakness and resultant respiratory insufficiency with hypoxia. Repetitive blood drawing (approximately 142 ml whole blood drawn per animal) hemodilution and other experimental manipulation all may have contributed to the decrease in hemoblobin. Other effects such as extravascular hemolysis cannot be ruled out and could have contributed to the elevated bilirubin.

### Example 12

In Study No. 4B, adult female Beagle dogs (one male in control group) were randomized into three study groups (3 animals/group) as shown in Table 17.

TABLE 17

| | | Dosage | | Dose | Number |
|---|---|---|---|---|---|
| Group | | Level | Concentration | Volume | of |
| No. | Treatment | (mg/kg) | (mg/ml) | (ml/kg) | Animals |
| 1 | Vehicle | 0 | 0 | 10 | 3 |
| | Control | | | | |
| 2 | β-Lapachone | 20 | 2 | 10 | 3 |
| 3 | β-Lapachone | 40 | 4 | 10 | 3 |

Animals were anesthetized with pentobarbital sodium (30 mg/kg IV bolus injection at a volume of 1 ml/kg followed by continuous infusion of 5 mg/kg/2.5 ml/hour IV throughout the experiment). β-lapachone dosing solutions were delivered by intravenous infusion over 1 hour via the cephalic vein. For measurement of cardiovascular function, a lead II ECG was obtained with subdermal needle electrodes and an ECG signal conditioner. Heart rate (HR) was measured with a pulse rate tachometer. The right femoral artery was cannulated with a catheter connected to a pressure transducer and pressure processor for recording mean, systolic and diastolic arterial blood pressure (BP). The left femoral artery was exposed by a flank incision and a probe (2.5 mm i.d.) connected to an electromagnetic flowmeter was placed around the artery for measurement of blood flow (BF). ECG, HR, BP, and BF were recorded and displayed on a thermal writing oscillograph. For measurement of pulmonary function, a 5.0 mm endotracheal tube connected to a pneumotachograph recorded integrated flow to yield a continuous recording of respiratory rate (RR). Intrapleural pressure was obtained from an esophageal balloon placed in the lower third of the esophagus. Transpulmonary pressure, the difference between thoracic (i.e. the external end of the endotracheal tube) and pleural pressure, was measured with a differential pressure transducer. Measurements of respiratory flow and transpulmonary pressure were used to compute total lung resistance (R_{L}) and dynamic compliance (C_{DYN}).

All parameters were measured and recorded immediately before (0 minute) and 5, 10, 30, 60, 90 and 120 minutes after start of the infusion. The mean±SEM value and the percentage relative to pretreatment values at each observation time point after dosing were calculated, and Dunnett's test was applied for comparison between vehicle control group and test substance treated group at the relative observation time interval. Differences were considered significant at p<0.05 level.

The only significant result in the 20 mg/kg dose group was slightly reduced tidal volume (16%) at 60 minutes, which reverted to normal by 120 minutes. β-lapachone at 40 mg/kg caused a 41% decrease in tidal volume by the end of the infusion accompanied by a four-fold increase in respiratory rate. Dynamic compliance was reduced to approximately 52% of normal and pulmonary resistance was increased approximately 50%. These changes all reverted toward normal during the recovery period. Neither dose of β-lapachone caused significant changes in blood pressure (mean, systolic or diastolic), blood flow, heart rate, or ECG (S-T segment, QRS duration, PR interval and QTc values).

The results of this study showed that intravenous infusions of β-lapachone up to and including 40 mg/kg over 1 hour have no significant effects on cardiovascular function in beagle dogs, and up to and including 20 mg/kg have no significant effects on pulmonary function. Administration of 40 mg/kg caused a reversible reduction in tidal volume and increase in respiratory rate which appear to be similar to the toxic signs seen in unanesthetized dogs and rats.

### Example 13

In Study No. 5B, three groups of three male Sprague-Dawley rats were dosed via the tail vein with 4×1 hour intravenous infusions at doses of 0 (vehicle), 30 and 45 mg/kg as shown in Table 18.

TABLE 18

| | | | | Number of |
|---|---|---|---|---|
| Group | Dosage Level | Concentration | Dose Volume | Animals |
| No. | (mg/kg) | (mg/ml) | (ml/kg) | (Male) |
| 1 | 0 | 0 | 15.0 | 3 |
| 2 | 30 | 2.0 | 15.0 | 3 |
| 3 | 45 | 3.0 | 15.0 | 3 |

Blood samples were taken for hematology and clinical chemistry (Days 1, 15 and 24) and coagulation (Day 24 only) and clinical chemistry evaluation.

No mortality or clinical signs of toxicity were noted in the control or 30 mg/kg dose groups. There was no mortality in the 45 mg/kg dose group, but clinical signs of toxicity were noted in one animal (noisy respiration, labored breathing, abnormal gait and stance) shortly after dose administration on Days 1 and 8 and resolved within 1-2 days. None of the animals exhibited signs following dosing on Days 15 or 22.

Hematology values were generally unremarkable in all groups except for a slightly elevated reticulocyte count in the high dose group on Day 15 (4.1% vs. 2.1% for controls). On Day 24, all hematology parameters including reticulocyte count were unremarkable. Serum clinical chemistry data showed slight increases in total bilirubin on Days 1 and 15 in Groups 2 and 3 (0.30 mg/dl-0.53 mg/dl with upper limit of normal 0.27 mg/dl), but normal in both groups on Day 24. Creatine kinase was elevated in both dosage groups (respectively 2-fold and 4-fold upper limit of normal) on Day 1 but normal by Day 15 despite continued dosing.

No abnormalities of internal organs were noted on necropsy. In the 45 mg/kg group, several minor histopathologic findings occurred of uncertain relevance. These included subtle bronchodilation and rare bronchial epithelial attenuation (also seen in some animals in Groups 1 and 2) and a slight degree of renal tubular epithelial swelling in two of three animals.

The results of this study showed that β-lapachone is well tolerated in male Sprague Dawley rats when administered as four weekly one-hour intravenous infusions at doses up to and including 45 mg/kg. Slight elevations in bilirubin on Days 1 and 15 (without indications of liver toxicity) and CK on Day 1 resolved by the end of the study despite continued dosing.

### Example 14

In Study No. 6B, two groups of three male Sprague-Dawley rats were dosed at 0 (vehicle) and 45 mg/kg as shown in Table 19.

TABLE 19

| | | | Dose | Number of |
|---|---|---|---|---|
| Group | Dosage Level | Concentration | Volume | Animals |
| Treatment | (mg/kg/day) | (mg/ml) | (ml/kg) | (Male) |
| Control | 0 | 0 | 15.0 | 3 |
| (Vehicle) | | | | |
| High-Dose | 45 | 3.0 | 15.0 | 3 |

The β-lapachone dosing solutions were administered daily for five consecutive days (Days 1 through 5) to each rat as an intravenous infusion over 1 hour into the tail vein. When no clinical signs of toxicity were noted after 5 doses, dose administration was increased to twice daily (approximately 8 hours between doses) and was continued through. Day 14. On Day 15 prior to sacrifice, blood samples were taken from all surviving animals for hematology, coagulation and serum clinical chemistry evaluation. Complete necropsies were performed, and major organs (adrenals, brain, heart, kidneys, lungs, spleen, liver and testes) were prepared for histopathological evaluation.

No mortality or clinical signs of toxicity were noted in the control group throughout the study or in the 45 mg/kg dose group through Day 5 except for an occasional observation of loose feces. On Day 9-10, animals began exhibiting rough hair coat, pale color, tail trauma related to drug administration, sporadic loose feces, and thin body condition. One animal was found in moribund condition on Day 13 and was euthanized. The two remaining β-lapachone-treated animals survived until sacrifice on Day 15.

Clinical laboratory determinations could be made on only one of the three 45 mg/kg animals (one animal found moribund, no sample obtained for another animal). Blood urea nitrogen was elevated (40 vs. ULN 19 mg/dl) with normal creatinine, and minor increases were present in total bilirubin (0.46 vs. ULN 0.27 mg/dL), and creatinine phosphokinase (553 vs. ULN 513 IU/L).

In the 45 mg/kg group, animals showed moderate to severe injection site injury including tail erosion and sloughing in the moribund animal.

On microscopic evaluation, the tail injection site and surrounding tissues showed moderate to severe inflammatory changes including soft tissue necrosis and vascular thrombosis in the lateral tail veins. Vascular thrombosis was also identified in two control animals but was not accompanied by necrosis and showed signs of healing.

In the animal sacrificed moribund, a thromboembolus found in the pulmonary artery likely caused or contributed to the animal's moribund state, although it was not obstructive. The thrombus most likely entered the systemic circulation via dislodgment from the site of vascular injury (lateral tail vein).

Test-article related splenic microscopic findings included mild to moderate lymphoid atrophy, histiocytosis with hemosiderosis, moderate congestion, and a mild increase in extramedullary hematopoiesis (EMH) in one animal compatible with a direct test-article effect or an indirect effect associated with the chronic inflammation induced at the injection site.

The results of this study showed that repetitive daily dosing of β-lapachone at 45 mg/kg up to twice daily for 14 days to male Sprague Dawley rats resulted in clinical signs of toxicity including lower body weight gains and food consumption. Microscopic findings were limited to the spleen, without test-article related changes in other organs.

### Example 15

Exponentially growing cells were seeded at 250, 1000, or 5000 cells per well (2.5 ml) in six-well plates and allowed to attach for 24 hours. β-lapachone was dissolved at a concentration of 20 mM in DMSO and diluted in complete media. β-lapachone (0.5 ml) was added at 6-fold the final concentration to a total volume of 3.0 ml/well. Control plates received the same volume of DMSO alone. After a 4 hour exposure the drug was carefully removed, and drug-free medium could be added. Cultures were left undisturbed for 14-21 days to allow for colony formation and then were fixed and stained with crystal violet stain (Sigma). Colonies of greater than 50 cells were scored as survivors. Cells were maintained at 37° C. in 5% CO₂ in complete humidity.

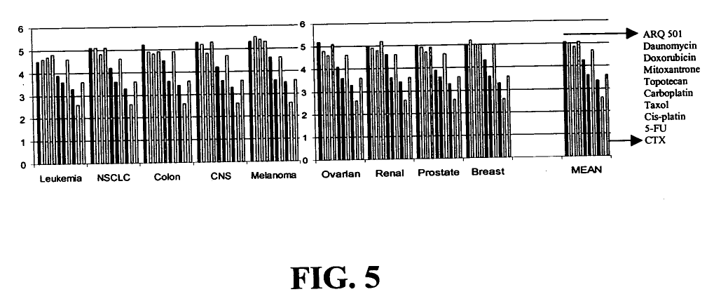

β-lapachone was tested in the NCI in vitro screen of 60 cancer cell lines, which allowed comparison with other anti-tumor agents under standardized conditions. The NCI screen includes cell lines isolated from leukemia, non-small cell lung cancer (NSCLC), colon, cerebral nervous system (CNS), melanoma, ovarian, renal, prostate and breast cancer tissues. The NCI assays were performed under standardized conditions and use the sulforhodamine B assay as the endpoint. β-lapachone is broadly active against many cell types, with LC₅₀ (log10 molar concentration causing 50% lethality) between −4.5 and −5.3, and mean of −5.07 across all cells. As shown in FIG. 5, when compared to many FDA approved chemotherapeutic agents for common cancer types with publicly available data, no approved drug exceeded the mean of β-lapachone across all cells and only mitoxantrone equaled it. Further, Table 20 showed the LC₅₀ (IM) for cell lines isolated from pancreatic, colon, prostate, ovarian, lung, breast and melanoma cancer tissues. Each “Replicate Result” represents the result of a separate experiment.

TABLE 20

| | | LC₅₀ (μM) |
|---|---|---|
| Tissue Origin | Cell Line | (Replicate Results) |
| Pancreatic | PaCa-2 | 1.68, 1.65, 1.52 |
| | BxPC-3 | 1.89, 2.09 |
| Colon | HT-29 | 2.19, 2.07, 1.99 |
| | HCT-116 | 2.29, 2.13 |
| Prostate | PC-3 | 2.13, 2.34 |
| | DU-145 | 1.10, 1.21 |
| Ovarian | SK-OV-3 | 1.18, 1.29 |
| | CAOV-3 | 1.56, 1.44 |
| Lung | A549 | 2.07, 1.81, 1.79 |
| Breast | MCF-7 | 1.45, 1.56 |
| Melanoma | SK-MEL-28 | 2.40, 2.63 |

### Example 16

Exponentially growing cells were seeded at 250, 1000, or 5000 cells per well (2.5 ml) in six-well plates and allowed to attach for 24 hours. β-lapachone was dissolved at a concentration of 20 mM in DMSO and diluted in complete media. β-lapachone (0.5 ml) was added at 6-fold the final concentration to a total volume of 3.0 ml/well. Control plates received the same volume of DMSO alone. After a 4 hour exposure the drug was carefully removed, and drug-free medium was added. Cultures were left undisturbed for 14-21 days to allow for colony formation and then were fixed and stained with crystal violet stain (Sigma). Colonies of greater than 50 cells were scored as survivors. Cells were maintained at 37° C. in 5% CO₂ in complete humidity. The results of three replicates are shown in Table 21. Each “Replicate Result” represents the result of a separate experiment.

TABLE 21

| | | LC₅₀ (μM) |
|---|---|---|
| Tissue Origin | Cell Line | (Replicate Results) |
| Colon | HT-29 | 2.19, 2.07, 1.99 |
| Colon | HCT-116 | 2.29, 2.13 |

### Example 17

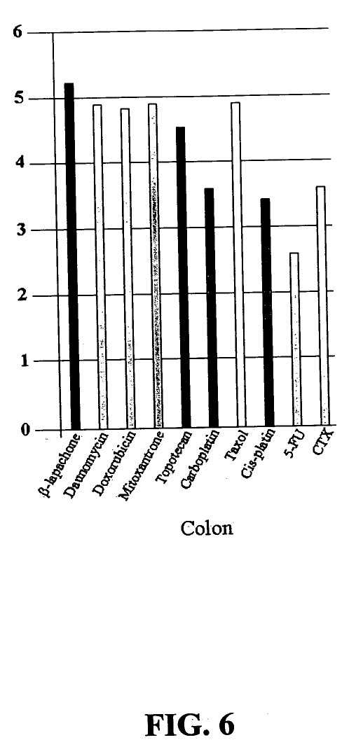

β-lapachone was tested in the NCI in vitro screen of 60 cancer cell lines, which allows comparison with other anti-tumor agents under standardized conditions. The NCI assays were performed under standardized conditions and use the sulforhodamine B assay as the endpoint. The NCI set of 60 lines included nine colon cancer cell lines (COLO 205, DLD-1, HCC-2998, HCT-116, HCT-15, HT29, KM12, KM20L2, and SW-620). The results show that β-lapachone was broadly active against many cell types, with LC₅₀ (log10 molar concentration causing 50% lethality) between −4.5 and −5.3, and mean of −5.07 across all cells. As shown in FIG. 6, when compared to many FDA approved chemotherapeutic agents for common cancer types with publicly available data, none of the compared approved drugs exceed the mean of β-lapachone across all cells and only mitoxantrone equals it.

### Example 18

Compounds of the present invention demonstrate potent antiproliferative activity against a variety of colon cancer cell lines, including SW-480, HT-29, DLD1 and HCT-116 human colon carcinoma cells. Since β-lapachone selectively induces apoptosis in cancer cell lines and not in normal cells (Li et al., (2003) Proc Natl Acad Sci USA. 100(5): 2674-8), the present compounds were also tested in a panel of normal cell lines from a variety of tissues including NCM 460 normal colonic epithelial cells. Cell proliferation assays are performed as described previously (Müller et al., (1996) J. Med. Chem. 39: 3132-3138; Müller et al., (1994) J. Med. Chem. 37: 1660-1669).

More specifically, exponentially growing cells were seeded at 1,000 cells per well in six-well plates and allowed to attach for 24 h. β-lapachone was solubilized in DMSO and was added to the wells in micromolar concentrations. Control wells were treated with equivalent volumes of DMSO. After 4 hours, the supernatant was removed and fresh medium was added. Cultures were observed daily for 10-15 days and then were fixed and stained. Colonies of greater than 30 cells were scored as survivors. The assays of the present invention are shown in Table 21 and methods of measuring induction of E2F activity and elevation of E2F levels were carried out following the descriptions found in Li et al., (2003) Proc Natl Acad Sci USA. 100(5): 2674-8 and U.S. Patent Application Publication No. 2002/0169135.

Table 22 shows the concentrations of the compounds that inhibit 50% of cell growth (IC₅₀). IC₅₀ values in the low micromolar range and below were obtained for β-lapachone in three colon cancer cell lines tested. Another effect of the compounds of the present invention is the induction or elevation of activity (e.g. elevation of the level) of at least one member of the E2F family of transcription factors (See, Table 22). The tested compounds of the present invention do not exhibit apparent or significant toxic effects on normal colon cells in the assays utilized.

TABLE 22

| | | | | Average | | |
|---|---|---|---|---|---|---|
| | | | | IC₅₀ | | |
| | Average IC₅₀ | | | Normal | | |
| | Colon Cancer Cell Lines | | | Cell lines | | |
| | (μM) | | | (μM) | E2F1 Induction | |
| | Colon | Colon | Colon | Colon | Colon | Colon |
| Compound | DLD1 | SW480 | HT-29 | NCM460 | SW480 | HT-29 |
| β-lapachone | 3.6 | 3.3 | 2.1 | 8.1 | + | + |

“+” symbol represents overexpression of E2F1 relative to a control.

### Example 19

Anti-tumor activity of β-lapachone was examined using a human colon cancer xenograft model. Athymic female nude mice (Ncr) were inoculated subcutaneously with 2×10⁶ HT-29 human colon cancer cells, and the tumors were allowed to grow to 80 mm3 in size. The animals were randomized into three groups. Animals were treated intraperitoneally every three days with either β-lapachone (60 mg/kg), 5-fluorouracil (5-FU, 40 mg/kg) or vehicle control, for a total of 10 treatments per animal, or daily with β-lapachone (40 mg/kg) for 28 days. Mean tumor volume was then analyzed.

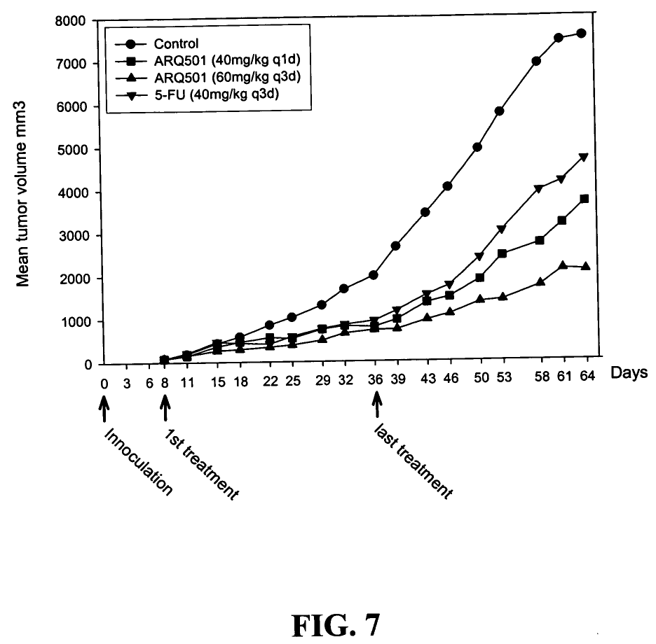

FIG. 7 shows that treatment with β-lapachone at 60 mg/kg reduced the mean tumor volume of xenografted human colon cancer by approximately 75%. No sign of significant toxicity was noted for any of the treatment regimens. In vitro experiments using cell lines of various tissue origins further showed that β-lapachone is non-toxic to normal cells.

Statistic significant tested by student's T tester (P value) are shown in Table 23.

TABLE 23

| | Con- | β-Lapachone | β-Lapachone | 5-Fu |
|---|---|---|---|---|
| | trol | 40 mg/kg q1d | 60 mg/kg q3d | 40 mg/kg q3d |
| Control | | P = 0.004514 | P = 0.000243 | P = 0.026868 |
| β-Lapachone | | | P = 0.114897 | P = 0.334636 |
| 40 mg/kg q1d | | | | |
| β-Lapachone | | | | P = 0.01403 |
| 60 mg/kg q3d | | | | |

### Example 20

Exponentially growing cells were seeded at 1000 per well in six-well plates and allowed to attach for 48 hours. Drugs were added to dishes in less than 5 μl of concentrated solution (corresponding to a final DMSO concentration of less that 0.1%). β-lapachone was dissolved at a concentration of 20 mM in DMSO and diluted in complete media. Control plates received the same volume of DMSO alone. After 1-4 hours exposure, cells were rinsed and drug-free medium was added. Cultures were left undisturbed for 10-20 days to allow for colony formation and then were fixed and stained with modified Wright-Giemsa stain (Sigma). Colonies of greater than 30 cells were scored as survivors. Cells were maintained at 37° C. in 5% CO₂ in complete humidity.

Treatment of G480 lung cancer cells for 4 hours with β-lapachone at 4 μM resulted in 68% cell death (i.e., survival of 32% of cells) in comparison to treatment with carrier alone. Table 23 provides a summary of the results. The number of colonies in control well was taken as 100% survival. Treated wells were presented as percentage of control. Data were presented as an average (+SEM) from three independent experiments.

TABLE 23

| | | Colonies (percent control) | | |
|---|---|---|---|---|
| Tissue | Cell | | | β-lapachone + |
| Origin | Line | β-lapachone | Taxol ® | Taxol ® |
| Lung | G480 | 32 (0.3) | 39 (2.6) | 2 (0.1) |

### Example 21

Exponentially growing cells were seeded at 250, 1000, or 5000 cells per well (2.5 ml) in six-well plates and allowed to attach for 24 hours. β-lapachone was dissolved at a concentration of 20 mM in DMSO and diluted in complete media. β-lapachone (0.5 ml) was added at 6-fold the final concentration to a total volume of 3.0 ml/well. Control plates received the same volume of DMSO alone. After a 4 hour exposure the drug was carefully removed, and drug-free medium was added. Cultures were left undisturbed for 14-21 days to allow for colony formation and then were fixed and stained with crystal violet stain (Sigma). Colonies of greater than 50 cells were scored as survivors. Cells were maintained at 37° C. in 5% CO₂ in complete humidity. The results of three replicates are provided in the Table 24. Each “Replicate Result” represents the result of a separate experiment.

TABLE 24

| | | LC₅₀ (μM) |
|---|---|---|
| Tissue Origin | Cell Line | (Replicate Results) |
| Lung | A549 | 2.07 |
| Lung | A549 | 1.81 |
| Lung | A549 | 1.79 |

### Example 22

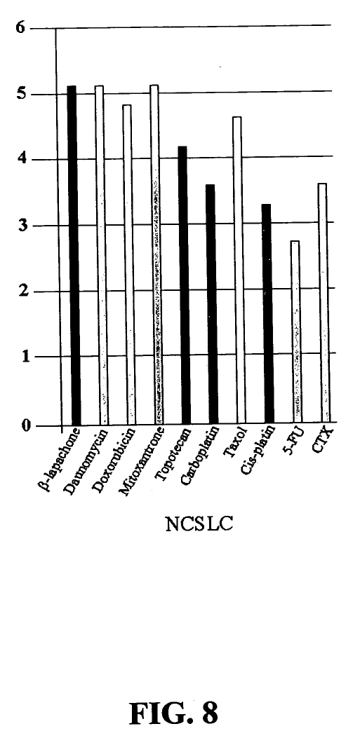

β-lapachone was tested in the NCI in vitro screen of 60 cancer cell lines, which allows comparison with other anti-tumor agents under standardized conditions. The NCI assays were performed under standardized conditions and use the sulforhodamine B assay as the endpoint. The NCI set of 60 lines included twelve non-small cell lung cancer cell lines (A549, EKVX, HOP-18, HOP-19, HOP-62, HOP-92, NCI-H226, NCI-H23, NCI-H322M, NCI-H460, NCI-H522, and LXFL 529), and three small cell lung cancer cell lines (DMS 114, DMS 273, AND SHP-77). The results show that β-lapachone was broadly active against many cell types, with LC₅₀ (log10 molar concentration causing 50% lethality) between −4.5 and −5.3, and mean of −5.07 across all cells. FIG. 8 shows that when compared to many FDA approved chemotherapeutic agents for common cancer types with publicly available data, none of the compared approved drugs exceed the mean of β-lapachone across all cells and only mitoxantrone equals it.

### Example 23

Exponentially growing A549 lung cancer cells were plated at 2×10⁵ cells in 60-mm dishes and allowed to attach for 48 hours. β-lapachone was dissolved at a concentration of 20 mM in DMSO and diluted in complete media. Growth media was removed from the cultures and β-lapachone was added at final drug concentrations of 1, 2, 5, 10 and 20 μM. After a 4 hour exposure, the drug media was aspirated and the cultures were washed with PBS, trypsinized, and plated at 20040,000 cells/100-mm dish. Variable cell numbers were plated to yield approximately 50-200 colonies/drug concentration. Cultures were left undisturbed for 14-21 days to allow for colony formation and were fixed and stained with crystal violet stain. Colonies of greater than 50 cells were scored as survivors. For each cell line, two colonies (“Clone A” and “Clone B”) were selected from those surviving >LC₉₉ concentrations of β-lapachone were isolated, expanded and used to repeat the assay. Individual cancer cells surviving 4-h exposures to >LC₉₉ concentrations of β-lapachone were isolated and cultured, then retested for sensitivity to β-lapachone.

As shown in Table 25 below, exponentially growing cultures of surviving lung cancer cells show the same LC₅₀ concentrations as the initial cultures. Attempts to generate resistance by long term continuous exposure of tumor cell lines to sublethal concentrations of β-lapachone has thus far also been unsuccessful. In vitro studies therefore suggest that β-lapachone does not select for resistant cell populations.

TABLE 25

| Tissue Origin | Cell Line | LC₅₀, μM |
|---|---|---|
| Lung | A549 WT | 1.79 |
| | Clone A | 1.69 |
| | Clone B | 1.50 |

### Example 24

The anti-tumor activity of β-lapachone was examined using a human lung cancer xenograft model. Athymic female nude mice (Ncr) were inoculated subcutaneously with 4×10⁶ A549 human lung cancer cells, and the tumors were allowed to grow to 50 mm³ in size. The animals were randomized into three groups of seven animals per group. Animals were treated intraperitoneally every three days with either β-lapachone (40 mg/kg or 60 mg/kg) or vehicle control, for a total of 8 treatments per animal. Mean tumor volume was then analyzed.

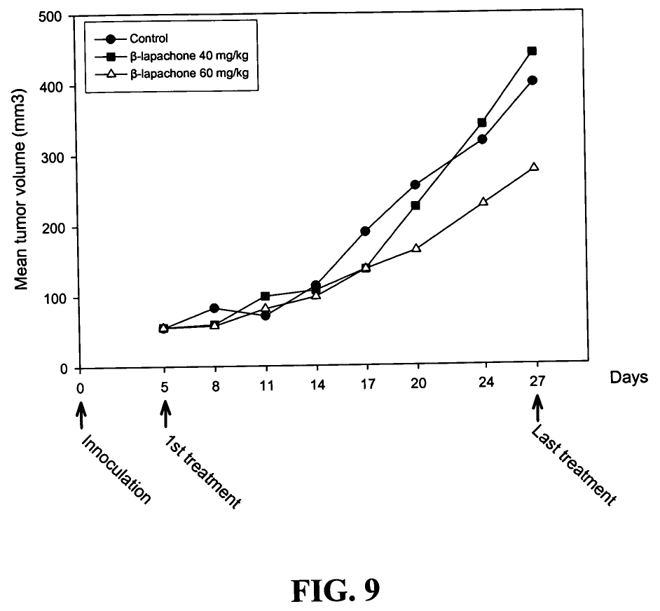

FIG. 9 shows that treatment with β-lapachone at 60 mg/kg reduced the mean tumor volume of xenografted human lung cancer by approximately 50%. No sign of significant toxicity was noted for any of the treatment regimens. In vitro experiments using cell lines of various tissue origins further showed that β-lapachone is relatively non-toxic to normal cells.

### Example 25

Micromolar concentrations of β-lapachone were shown to totally abolish colony formation when applied to tumor cell cultures in combination with IC₅₀ levels of Taxol®. In these studies, exponentially growing cells were seeded at 1,000 cells per well in six-well plates and allowed to attach for 48 h. β-lapachone and/or Taxol®, solubilized in DMSO, were added to the wells. Control wells were treated with equivalent volumes of DMSO. After 4 hours cells were rinsed and fresh medium is added. Cultures were observed daily for 10-20 days and then were fixed and stained. Colonies of greater than 30 cells were scored as survivors. As shown in Table 26, synergistic inhibition of cancer cell survival is seen for a human carcinoma pancreatic cell line. The decreased cell survival was shown to be due to death by the MTT and tryptan blue exclusion assays. DNA laddering formation and annexin staining indicate that cell death is due to apoptosis.

TABLE 26

| | | | | Colonies, Percentage of control | | |
|---|---|---|---|---|---|---|
| | Tissue | Applied Drug conc., μM | | | | β-Lapachone + |
| Cell Line | Origin | β-Lapachone | Taxol ® | β-Lapachone | Taxol ® | Taxol ® |
| ASPC-1 | Pancreas | 4 | 0.2 | 45 (1.9) | 71 (0.8) | 0 |

### Example 26

Exponentially growing cells were seeded at 250, 1000, or 5000 cells per well (2.5 ml) in six-well plates and allowed to attach for 24 hours. β-lapachone was dissolved at a concentration of 20 mM in DMSO and diluted in complete media. β-lapachone (0.5 ml) was added at 6-fold the final concentration to a total volume of 3.0 ml/well. Control plates receive the same volume of DMSO alone. After a 4 hour exposure the drug was carefully removed, and drug-free medium was added. Cultures were left undisturbed for 14-21 days to allow for colony formation and then were fixed and stained with crystal violet stain (Sigma). Colonies of greater than 50 cells were scored as survivors. Cells were maintained at 37° C. in 5% CO₂ in complete humidity. The results of three replicates are provided in Table 27. Each “Replicate Result” represents the result of a separate experiment.

TABLE 27

| | | LC₅₀ (μM) |
|---|---|---|
| Tissue Origin | Cell Line | (Replicate Results) |
| Pancreatic | PaCa-2 | 1.68 |
| Pancreatic | PaCa-2 | 1.65 |
| Pancreatic | PaCa-2 | 1.52 |
| Pancreatic | BxPC-3 | 1.89 |
| Pancreatic | BxPC-3 | 2.09 |

### Example 27

Exponentially growing PaCa-2 pancreatic cancer cells were plated at 2×10⁵ cells in 60-mm dishes and allowed to attach for 48 hours. β-lapachone was dissolved at a concentration of 20 mM in DMSO and diluted in complete media. Growth media was removed from the cultures and β-lapachone was added at final drug concentrations of 1, 2, 5, 10 and 20 μM. After a 4 hour exposure, the drug media was aspirated and the cultures were washed with PBS, trypsinized, and plated at 200-40,000 cells/100-mm dish. Variable cell numbers were plated to yield approximately 50-200 colonies/drug concentration. Cultures were left undisturbed for 14-21 days to allow for colony formation and were fixed and stained with crystal violet stain. Colonies of greater than 50 cells were scored as survivors. For each cell line, two colonies (“Clone A” and “Clone B”) were selected from those surviving >LC₉₉ concentrations of β-lapachone were isolated, expanded and used to repeat the assay. Individual cancer cells surviving 4-h exposures to >LC₉₉ concentrations of β-lapachone were isolated and cultured, then retested for sensitivity to β-lapachone.

As shown in Table 28, exponentially growing cultures of surviving pancreatic cancer cells show the same LC₅₀ concentrations as the initial cultures. Attempts to generate resistance by long term continuous exposure of tumor cell lines to sublethal concentrations of β-lapachone has thus far also been unsuccessful. In vitro studies therefore suggest that β-lapachone does not select for resistant cell populations.

TABLE 28

| Tissue Origin | Cell Line | LC₅₀, μM |
|---|---|---|
| Pancreatic | PaCa-2 WT | 2.69 |
| | Clone A | 2.59 |
| | Clone B | 2.72 |

### Example 28

Compounds of the present invention demonstrate potent antiproliferative activity against a variety of cancer cell lines, including MIA PACA-2 and BXPC-3 human pancreatic carcinoma cells. Exponentially growing cells were seeded at 1,000 cells per well in six-well plates and allowed to attach for 24 h. β-lapachone was solubilized in DMSO and was added to the wells in micromolar concentrations. Control wells were treated with equivalent volumes of DMSO. After 4 hours, the supernatant was removed and fresh medium was added. Cultures were observed daily for 10-15 days and then were fixed and stained. Colonies of greater than 30 cells were scored as survivors.

Table 29 shows the concentrations of the compounds required to inhibit 50% of cell growth (IC₅₀). IC₅₀ values in the low micromolar range and below were obtained for β-lapachone in a pancreatic cancer cell line. Another effect of the compounds of the present invention is the induction or elevation of activity (e.g., elevation of the level) of at least one member of the E2F family of transcription factors Studies have shown that β-lapachone induces sustained E2F activity (e.g. elevation of E2F levels) in nuclei of cancer cells but not in normal cells, resulting in the arrest of cancer cells in G1 and/or S phase. β-lapachone was effective in inducing E2F activity (e.g. elevating E2F levels), thus causing G1 and/or S phase arrest.

TABLE 29

| | Average IC₅₀ | | | Average IC₅₀ | |
|---|---|---|---|---|---|
| | Cancer Cell Lines | | | Normal | E2F1 |
| | (μM) | | | Colon (μM) | Induction |
| | Pancreas | Colon | Colon | Colon | Pancreas |
| Compound | PaCa-2 | SW480 | HT-29 | NCM460 | PANC1 |
| β-lapachone | 1.6 | 3.3 | 2.1 | 8.1 | + |

“+” symbol represents overexpression of E2F1 relative to a control.

### Example 29

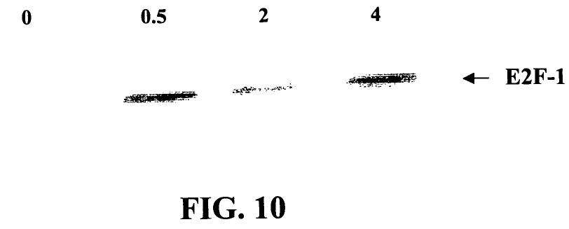

FIG. 10 shows that E2F-1 protein expression was upregulated by β-Lapachone in human pancreatic cancer cells (Paca-2), as demonstrated by Western blot analysis. In this experiment, Paca-2 cells were seeded in medium and exposed for 0.5 hours to 0 (vehicle), 0.5, 2 or 4 μM concentrations of β-Lapachone. Cells were harvested and whole cell lysates were prepared and resolved by SDS/PAGE, then Western blots were prepared using E2F-1 antibody obtained from Santa Cruz Biotechnology (Santa Cruz, Calif.) and an enhanced chemiluminescence assay system (Amersham Pharmacia). The blot shows that E2F-1 protein is induced by the lowest concentration of β-Lapachone tested, 0.5 μM.

### Example 30

The anti-tumor activity of β-lapachone was examined using a human pancreatic cancer xenograft model. Athymic female nude mice (Ncr) were inoculated subcutaneously with 4×10⁶ human Panc-1 pancreatic cancer cells, and the tumors were allowed to grow to 50 mm3 in size. The animals were randomized into three groups. Animals were treated intraperitoneally every three days with either β-lapachone (40 mg/kg or 60 mg/kg) or vehicle control, for a total of 5 treatments per animal. Mean tumor volume was then analyzed.

The studied showed that treatment with β-lapachone at 40 and 60 mg/kg reduced the mean tumor volume of xenografted human pancreatic cancer. No sign of significant toxicity was noted for any of the treatment regimens. In vitro experiments using cell lines of various tissue origins further showed that β-lapachone is relatively non-toxic to normal cells.

### Example 31

Proliferating human leukemia or lymphoma cells were seeded at 1000 per well in six-well plates and incubated 48 hours. β-lapachone was added to dishes in less than 5 μl of concentrated solution (corresponding to a final DMSO concentration of less that 0.1%). β-lapachone was dissolved at a concentration of 20 mM in DMSO and diluted in complete media. Control plates received the same volume of DMSO alone. After 1-4 hours exposure, cells were rinsed and drug-free medium could be added. Cultures were left undisturbed for 10-20 days to allow for colony formation and then could be fixed and stained with modified right-Giemsa stain (Sigma). Colonies of greater than 30 cells were scored as survivors. Cells were maintained at 37° C. in 5% CO₂ in complete humidity.

Alternatively, cell death of human leukemia or lymphoma cells cultured in the absence or presence of β-Lapachone (e.g., at 2, 4, 8, and 20 μM) for one to 24 hours was measured by MTT assay. Briefly, the MTT assay was performed by plating in a 96-well plate at 10,000 cells per well, culturing for 48 hours in complete growth-medium, treating with β-lapachone for one to 24 hours, and cultured with drug-free medium for 24 hours. MTT solution was added to the culture medium, and after 2 hours, optical density could be read with an ELISA reader.

Alternatively, cell death of human leukemia or lymphoma cells cultured in the absence or presence of β-Lapachone was measured by fluoresence activated cell sorting (FACS) analysis. Alternatively, cell death of human leukemia or lymphoma cells cultured in the absence or presence of β-lapachone was measured by methods described in Li et al., (2003) Proc Natl Acad Sci USA. 100(5): 2674-8.

### Example 32

Androgen-independent, p53 null DU145 cells (8×10⁶) were inoculated subcutaneously into male SCID (ICR) mice and observed for approximately 21 days to allow inocula to develop into palpable tumors approximately 5 mm in diameter. Animals were then treated intraperitoneally with β-lapachone or vehicle control every three days for 21-30 days. Following termination of treatment, animals were observed for an additional 14 days. Tumors were measured throughout treatment and the post-treatment observation period.

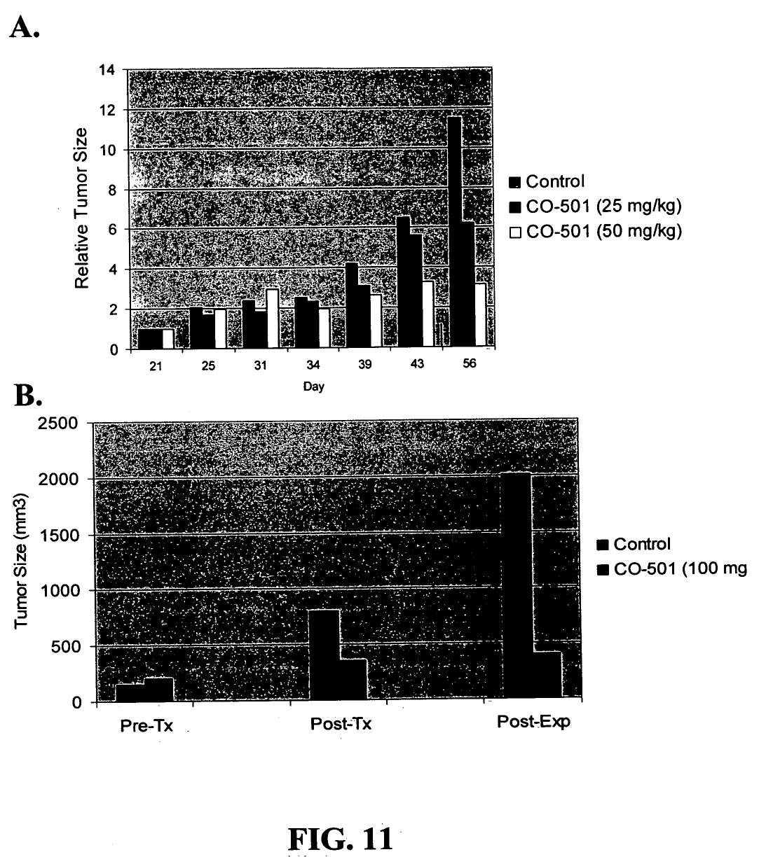

In FIG. 11A, SCID (ICR) mice with established subcutaneous DU145 human prostate cancer were given β-lapachone (25 or 50 mg/kg in Lipiodol IP q3d) or vehicle control on a similar schedule. Tumor nodules were measured during the treatment period (Days 21-43) and at the end of observation (Day 56). Dose-related inhibition of tumor growth is seen, with little tumor growth following completion of treatment. As shown in FIG. 11A, treatment with 25 and 50 mg/kg β-lapachone inhibited tumor growth in a dose-dependent manner, with little growth of tumors in the 2 week period following termination of treatment.

In FIG. 11B, SCID (ICR) mice with established subcutaneous DU145 human prostate cancer were given β-lapachone (100 mg/kg in Lipiodol IP q3d) or vehicle control on a similar schedule. Tumor nodules were measured pre-treatment (“Pre-Tx”), at the end of the 30-day treatment period (“Post-Tx”) and at the conclusion of the post treatment observation period 14 days following termination of treatment (“Post-Exp't”). FIG. 11B shows that greater suppression of tumor growth was observed when β-lapachone was given to established tumors at 100 mg/kg in a separate experiment of similar design.

### Example 33

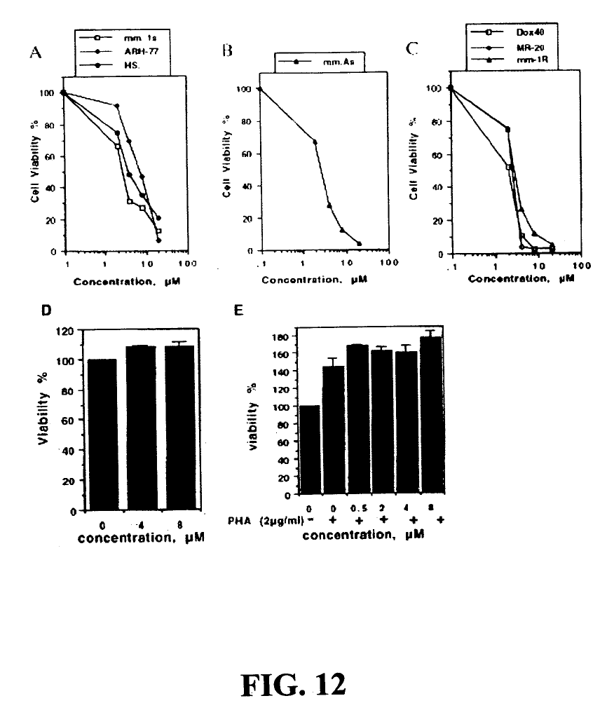

10 Studies have shown that β-Lapachone modulates (i.e. activates or inhibits) checkpoints and induces apoptosis in cancer cells from a variety of tissues without affecting normal cells from these tissues (U.S. Publication No. US-2002-0169135-A1). In this study, proliferation of multiple myeloma (MM) cells cultured in the absence or presence of β-Lapachone (2, 4, 8, and 20 μM) for 24 h was measured by MTT assay. At a concentration of 4 μM, cell viability in cultures was found to be significantly decreased in all seven MM cell lines, including dramatic reduction in the proliferation of a patient's MM cells and drug-resistant cells. To investigate the cytotoxicity of β-Lapachone on human PBMC, cells were isolated from anticoagulant-treated blood. Proliferating PBMC were generated by 72 h incubation with phytohemagglutinin (PHA) at 2 μg/mL. Growth of cells culture in the absence or presence of β-Lapachone (0.5, 2, 4, and 8 μM) for 24 h was measured by MT. No cytotoxicity to either fresh or proliferating PBMC growth was observed. FIG. 12 shows the differential effects of β-lapachone on human multiple myeloma (MM) cells vs. normal human Peripheral Blood Mononuclear Cells (PBMC).

### Example 34

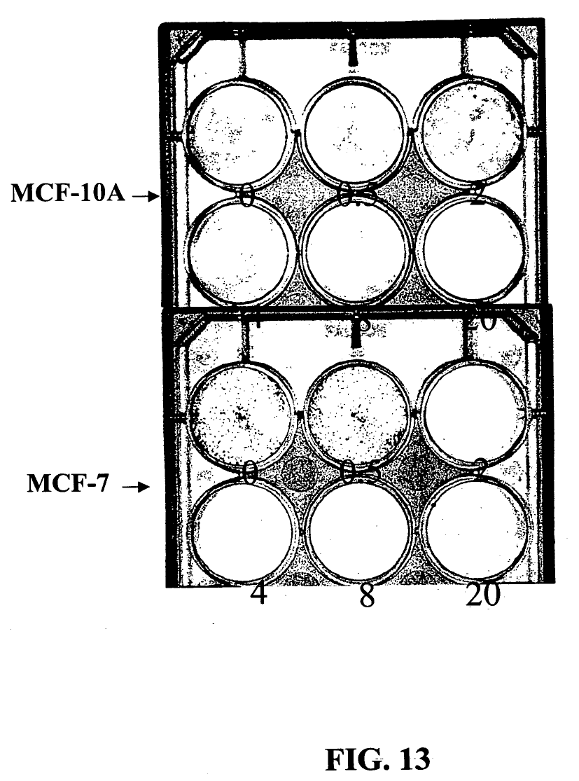

In this study, exponentially growing cells were seeded at 1000 cells/well and allowed to attach for 48 h. The cells were treated for 4 h with β-Lapachone at various concentrations, then were rinsed and fresh medium was added. After 10-20 days, cells were fixed and stained with modified Wright-Giemsa stain. The human breast cancer cells (MCF-7) show essentially complete elimination of colonies at β-Lapachone concentrations of 24 μM and higher, whereas the normal breast epithelial cells (MCF-10A) show no reduction in the number of colonies, although the size of the colonies is smaller, as would be expected by checkpoint activated growth delay. FIG. 13 shows the differential effects of β-Lapachone (PIM) on human breast cancer cells (MCF-7) vs. normal human breast epithelial cells (MCF-10A).

1. A method of treating cancer or a precancerous condition or preventing cancer in a subject, the method comprising administering to the subject a therapeutically effective amount of a pharmaceutical composition comprising β-lapachone, or a derivative or analog thereof, or pharmaceutically acceptable salt thereof, or a metabolite thereof, and a pharmaceutically acceptable carrier such that said composition maintains a plasma concentration of about 0.15 μM to about 50 μM and treats said cancer or precancerous condition or prevents said cancer.

2. A method of treating cancer or a precancerous condition or preventing cancer in a subject, the method comprising administering to the subject a therapeutically effective amount of a pharmaceutical composition comprising β-lapachone, or a derivative or analog thereof, or pharmaceutically acceptable salt thereof, or a metabolite thereof, and a pharmaceutically acceptable carrier such that said composition maintains a plasma concentration of about 0.15 μM to about 50 μM, modulates one or more cell cycle checkpoints in one or more cancer cells and treats said cancer or precancerous condition or prevents said cancer.

3. A method of treating cancer or a precancerous condition or preventing cancer in a subject, the method comprising administering to the subject a therapeutically effective amount of a pharmaceutical composition comprising β-lapachone, or a derivative or analog thereof, or pharmaceutically acceptable salt thereof, or a metabolite thereof, and a pharmaceutically acceptable carrier such that said composition maintains a plasma concentration of about 0.15 μM to about 50 μM, modulates cell death selectively in one or more cancer cells and treats said cancer or precancerous condition or prevents said cancer.

4. A method of treating or preventing a cell proliferative disorder in a subject, the method comprising administering to the subject a therapeutically effective amount of a pharmaceutical composition comprising β-lapachone, or a derivative or analog thereof, or pharmaceutically acceptable salt thereof, or a metabolite thereof, and a pharmaceutically acceptable carrier such that said composition maintains a plasma concentration of about 0.15 μM to about 50 μM and treats or prevents said cell proliferative disorder.

5. The method claim 2, wherein the activation of one or more cell cycle checkpoints modulates one or more cell cycle pathways in one or more cancer cells.

6. The method claim 2, wherein the activation of one or more cell cycle checkpoints activates one or more cell cycle regulators in one or more cancer cells.

7. The method of claims 1-4, wherein the plasma concentration is about 0.175 μM to about 30 μM.

8. The method of claims 1-4, wherein the plasma concentration is about 0.2 μM to about 20 μM.

9. The method of claims 1-4, wherein the subject is exposed to the pharmaceutical composition in an AUC range of about 0.5 μM-hr to about 100 μm-hr.

10. The method of claims 1-4, wherein the subject is exposed to the pharmaceutical composition in an AUC range of about 1 μM-hr to about 25 μM-hr.

11. The method of claims 1-4, wherein the subject is exposed to the pharmaceutical composition in an AUC range of about 1.5 μM-hr to about 6.5 μM-hr.

12. The method of claims 1-4, wherein the pharmaceutical composition is administered at a dosage from about 2 mg/m² to 5000 mg/m² per day.

13. The method of claims 1-4, wherein the pharmaceutical composition is administered at a dosage from about 20 mg/m² to 2000 mg/m² per day.

14. The method of claims 1-4, wherein the pharmaceutical composition is administered at a dosage from about 30 to 300 mg/m² per day.

15. The method of claims 1-4, wherein the pharmaceutical composition is administered intravenously, orally or intraperitoneally.

16. The method of claims 1-3, wherein the cancer is selected from the group consisting of multiple myeloma, chronic myelogenous leukemia, pancreatic cancer, non-small cell lung cancer, lung cancer, breast cancer, colon cancer, ovarian cancer, prostate cancer, malignant melanoma, non-melanoma skin cancers, hematologic tumors, hematologic tumors, hematologic malignancies, childhood leukemia, childhood lymphomas, multiple myeloma, Hodgkin's disease, lymphomas of lymphocytic origin, lymphomas of cutaneous origin, acute leukemia, chronic leukemia, acute lymphoblastic leukemia, acute myelocytic leukemia, chronic myelocytic leukemia, plasma cell neoplasm, lymphoid neoplasm, cancers associated with AIDS, cancers of the tongue, mouth, pharynx, and oral cavity, esophageal cancer, stomach cancer, cancer of the small intestine, anal cancer, cancer of the anal canal, anorectal cancer, liver cancer, intrahepatic bile duct cancer, gallbladder cancer, biliary cancer, cancer of other digestive organs, cancer of the larynx, bone and joint cancer, uterine cancer, cervical cancer, uterine corpus cancer, cancer of the vulva, vaginal cancer, testicular cancer, penile cancer, urinary bladder cancer, kidney cancer, renal cancer, cancer of the ureter and other urinary organs, ocular cancer, brain and nervous system cancer, CNS cancers, and thyroid cancer.

17. The method of claims 1-4, wherein the pharmaceutically acceptable carrier is a solubilizing carrier molecule selected from the group consisting of Poloxamer, Povidone K17, Povidone K12, Tween 80, ethanol, Cremophor/ethanol, Lipiodol, polyethylene glycol (PEG) 400, propylene glycol, Trappsol, alpha-cyclodextrin or analogs thereof, beta-cyclodextrin or analogs thereof, and gamma-cyclodextrin or analogs thereof.

18. The method of claims 1-4, wherein the subject is a mammal.

19. The method of claim 18, wherein the subject is a human.

20. The method of claims 1-3, wherein treating said cancer comprises a reduction in tumor size.

21. The method of claims 1-3, wherein treating said cancer comprises a reduction in tumor number.

22. The method of claims 1-3, wherein treating said cancer comprises a decrease in tumor growth rate.

23. The method of claims 1-3, wherein treating said cancer comprises a decrease of tumor regrowth.

24. The method of claims 1-3, wherein treating said cancer comprises an increase in average survival time of a population of treated subjects in comparison to an untreated population.

25. The method of claims 1-3, wherein treating said cancer comprises an increase in average survival time of a population of treated subjects in comparison to a population receiving monotherapy with a drug that is not β-lapachone.

26. The method of claims 1-4, wherein administration results in activation of a cell cycle checkpoint.

27. The method of claim 26, wherein administration results in activation of a G1 or S cell cycle checkpoint.

28. The method of claims 14, wherein administration results in activation of an E2F transcription factor pathway.

29. The method of claims 14, wherein administration induces elevation of an E2F transcription factor.

30. The method of claims 14, wherein administration induces elevation of an E2F transcription factor selectively.

31. The method of claims 14, wherein administration stimulates unscheduled activation of an E2F transcription factor.

32. The method of claims 14, wherein administration stimulates unscheduled activation of an E2F transcription factor selectively.

33. The method of claims 1-4, wherein administration induces cell death selectively.

34. The method of claims 3 or 33, wherein said cell death is selected from the group consisting of apoptosis, necrosis and senescence.

35. The method of claims 1-4, wherein said therapeutically effective amount is not cytotoxic to normal cells

36. The method of claims 1-4, wherein said therapeutically effective amount does not affect normal cell viability

37. The method of claims 1-4, further comprising administering a therapeutically effective amount of a second anti-cancer agent or a second anti-proliferative agent, or a derivative or analog thereof.

38. The method of claim 37, wherein the second anti-cancer agent or anti-proliferative agent is selected from the group consisting of paclitaxel, docetaxel, vincristin, vinblastin, nocodazole, epothilones, navelbine, etoposide, adriamycin, camptothecin, daunorubicin, dactinomycin, mitoxantrone, amsacrine, epirubicin, idarubicin, gemcitabine and imatinib.

39. The method of claim 37, wherein the pharmaceutical composition is administered simultaneously with or following administration of the second anti-cancer agent or second anti-proliferative agent.

40. The method of claim 37, wherein the second anti-cancer agent or second anti-proliferative agent is administered following administration of the pharmaceutical composition.

41. The method of claim 37, wherein the second anti-cancer agent or second anti-proliferative agent is administered within 24 hours after the pharmaceutical composition is administered.
